(19) United States
(12) Patent Application Publication
(10) Pub. No.: US 2010/0226889 A1
(43) Pub. Date: Sep. 9, 2010

(54) HCV Combination Therapies

(75) Inventor: Lindsay McNair, Allston, MA (US)

Correspondence Address: Jonathan P. O''Brien, Ph.D.;Honigman Miller Schwartz & Cohn LLP, 350 East Michigan Avenue, Suite 300, Kalamazoo, MI, 49007, US

(73) Assignee: VERTEX PHARMACEUTICALS INCORPORATED, Cambridge, MA (US)

(21) Appl. No.: 12/773,364

(22) Filed: May 4, 2010

Related U.S. Application Data
(63) Continuation of application No. PCT/US2008/012460, filed on Nov. 4, 2008.
(60) Provisional application No. 60/985,522, filed on Nov. 5, 2007.
(60) Provisional application No. 61/085,145, filed on Jul. 31, 2008.
(60) Provisional application No. 61/109,690, filed on Oct. 30, 2008.

(51) Int. Cl. A61K 38/21; A61P 31/12

(52) U.S. Cl. 424/85.7

(57) ABSTRACT

The invention relates to combination therapies for the treatment of hepatitis C virus with telaprevir and pegylated interferon alfa-2a with or without ribavirin. The invention relates to the treatment of Latino and African American patients infected with HCV using the combination therapy.

### CROSS-REFERENCE

The present application claims priority to U.S. Application No. 60/985,522 filed on Nov. 5, 2007, U.S. Application No. 61/085,145, filed on Jul. 31, 2008, and U.S. Application No. 61/109,690 filed on Oct. 30, 2008, the contents of which are incorporated herein by reference in their entireties.

### TECHNICAL FIELD OF THE INVENTION

In general, the invention relates to combination therapies for the treatment of hepatitis C virus (“HCV”) with telaprevir (TVR, T or VX-950), an oral inhibitor of HCV protease, with pegylated interferon alfa-2a (peg-IFN or P) and/or ribavirin (RBV or R). The invention relates to the treatment of Latino and African American patients infected with HCV using the combination therapy.

### BACKGROUND OF THE INVENTION

Infection by HCV is a compelling human medical problem. HCV is recognized as the causative agent for most cases of non-A, non-B hepatitis, with an estimated human sero-prevalence of 3% globally [A. Alberti et al., “Natural History of Hepatitis C,” J. Hepatology, 31., (Suppl. 1), pp. 17-24 (1999)]. Nearly four million individuals may be infected in the United States alone [M. J. Alter et al., “The Epidemiology of Viral Hepatitis in the United States, Gastroenterol. Clin. North Am., 23, pp. 437-455 (1994); M. J. Alter “Hepatitis C Virus Infection in the United States,” J. Hepatology, 31., (Suppl. 1), pp. 88-91 (1999)].

Upon first exposure to HCV only about 20% of infected individuals develop acute clinical hepatitis while others appear to resolve the infection spontaneously. In almost 70% of instances, however, the virus establishes a chronic infection that persists for decades [S. Iwarson, “The Natural Course of Chronic Hepatitis,” FEMS Microbiology Reviews, 14, pp. 201-204 (1994); D. Lavanchy, “Global Surveillance and Control of Hepatitis C,” J. Viral Hepatitis, 6, pp.

35-47 (1999)]. This usually results in recurrent and progressively worsening liver inflammation, which often leads to more severe disease states such as cirrhosis and hepatocellular carcinoma [M. C. Kew, “Hepatitis C and Hepatocellular Carcinoma”, FEMS Microbiology Reviews, 14, pp. 211-220 (1994); I. Saito et. al., “Hepatitis C Virus Infection is Associated with the Development of Hepatocellular Carcinoma,” Proc. Natl. Acad. Sci. USA, 87, pp. 6547-6549 (1990)]. Unfortunately, there are no broadly effective treatments for the debilitating progression of chronic HCV.

The HCV genome encodes a polyprotein of 3010-3033 amino acids [Q. L. Choo, et. al., “Genetic Organization and Diversity of the Hepatitis C Virus.” Proc. Natl. Acad. Sci. USA, 88, pp. 2451-2455 (1991); N. Kato et al., “Molecular Cloning of the Human Hepatitis C Virus Genome From Japanese Patients with Non-A, Non-B Hepatitis,” Proc. Natl. Acad. Sci. USA, 87, pp. 9524-9528 (1990); A. Takamizawa et. al., “Structure and Organization of the Hepatitis C Virus Genome Isolated From Human Carriers,” J. Virol., 65, pp. 1105-1113 (1991)]. The HCV nonstructural (NS) proteins are presumed to provide the essential catalytic machinery for viral replication. The NS proteins are derived by proteolytic cleavage of the polyprotein [R. Bartenschlager et. al., “Nonstructural Protein 3 of the Hepatitis C Virus Encodes a Serine-Type Proteinase Required for Cleavage at the NS3/4 and NS4/5 Junctions,” 3. Virol., 67, pp. 3835-3844 (1993); A. Grakoui et. al., “Characterization of the Hepatitis C Virus-Encoded Serine Proteinase: Determination of Proteinase-Dependent Polyprotein Cleavage Sites,” J. Virol., 67, pp. 2832-2843 (1993); A. Grakoui et. al., “Expression and Identification of Hepatitis C Virus Polyprotein Cleavage Products,” J. Virol., 67, pp. 1385-1395 (1993); L. Tomei et. al., “NS3 is a serine protease required for processing of hepatitis C virus polyprotein”, J. Virol., 67, pp. 4017-4026 (1993)].

The HCV NS protein 3 (NS3) contains a serine protease activity that helps process the majority of the viral enzymes, and is thus considered essential for viral replication and infectivity. It is known that mutations in the yellow fever virus NS3 protease decrease viral infectivity [Chambers, T. J. et. al., “Evidence that the N-terminal Domain of Nonstructural Protein NS3 From Yellow Fever Virus is a Serine Protease Responsible for Site-Specific Cleavages in the Viral Polyprotein”, Proc. Natl. Acad. Sci. USA, 87, pp. 8898-8902 (1990)]. The first 181 amino acids of NS3 (residues 1027-1207 of the viral polyprotein) have been shown to contain the serine protease domain of NS3 that processes all four downstream sites of the HCV polyprotein [C. Lin et al., “Hepatitis C Virus NS3 Serine Proteinase: Trans-Cleavage Requirements and Processing Kinetics”, J. Virol., 68, pp. 8147-8157 (1994)].

The HCV NS3 serine protease and its associated cofactor, NS4A, help process all of the viral enzymes, and are thus considered essential for viral replication. This processing appears to be analogous to that carried out by the human immunodeficiency virus aspartyl protease, which is also involved in viral enzyme processing. HIV protease inhibitors, which inhibit viral protein processing, are potent antiviral agents in man indicating that interrupting this stage of the viral life cycle results in therapeutically active agents. Consequently HCV NS3 serine protease is also an attractive target for drug discovery.

Until recently, the only established therapy for HCV disease was interferon treatment. However, interferons have significant side effects [M. A. Walker et al., “Hepatitis C Virus: An Overview of Current Approaches and Progress,” DDT, 4, pp. 518-29 (1999); D. Moradpour et al., “Current and Evolving Therapies for Hepatitis C,” Eur. J. Gastroenterol. Hepatol., 11, pp. 1199-1202 (1999); H. L. A. Janssen et al. “Suicide Associated with Alfa-Interferon Therapy for Chronic Viral Hepatitis,” J. Hepatol., 21, pp. 241-243 (1994); P. F. Renault et al., “Side Effects of Alpha Interferon,” Seminars in Liver Disease, 9, pp. 273-277. (1989)] and induce long term remission in only a fraction (˜25%) of cases [O. Weiland, “Interferon Therapy in Chronic Hepatitis C Virus Infection”, FEMS Microbiol. Rev., 14, pp. 279-288 (1994)]. Recent introductions of the pegylated forms of interferon (PEG-INTRON® and PEGASYS®) and the combination therapy of ribavirin and pegylated interferon (REBETROL®) have resulted in only modest improvements in remission rates and only partial reduction of side effects. Moreover, the prospects for effective anti-HCV vaccines remain uncertain.

Previous prospective trials have reported lower response rates to HCV therapy among treatment-naïve subjects from different racial and ethnic groups. African Americans with genotype 1 HCV infection treated with peginterferon alfa and ribavirin achieved sustained virologic response (SVR) with rates of 19% and 28%. Among Latinos treated with Peg-IFN alfa-2a and RBV, 34% achieved SVR compared with 49% of Caucasians.

Thus, there is a need for more effective anti-HCV therapies. Such inhibitors would have therapeutic potential as protease inhibitors, particularly as serine protease inhibitors, and more particularly as HCV NS3 protease inhibitors. Specifically, such compounds may be useful as antiviral agents, particularly as anti-HCV agents.

VX-950, an HCV inhibitor with its structure shown below is such a compound in need. VX-950 is described in PCT Publication Number WO 02/18369, which is incorporated herein by reference in its entirety.

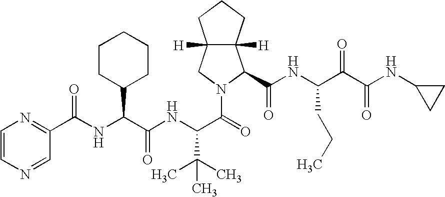

VX-950, a potent and specific NS3-4A protease inhibitor demonstrated substantial antiviral activity in a phase 1b trial of subjects infected with HCV genotype 1 (Study VX04-950-101). The degree to which a subject responds to treatment and the rate at which viral rebound is observed could in part be due to genotypic differences in sensitivity to the protease inhibitor. The rapid replication rate of HCV, along with the poor fidelity of its polymerase, gives rise to an accumulation of mutations throughout its genome [P. Simmonds, “Genetic diversity and evolution of hepatitis C virus—15 years on,” J. Gen. Virol. 85, pp. 3173-88 (2004)]. The degree to which sequence variability in the protease region affects the catalytic efficiency of the enzyme or the binding of an inhibitor is not known. Additionally, the generation of numerous viral genomes with remarkable sequence variation presents potential problems of emerging drug resistant virus in subjects treated with antiviral therapy. Indeed, drug resistance against antiviral drugs, such as HIV protease inhibitors, is well documented [Johnson, et al., Top. HIV Med. 12, pp. 119-24 (2004)]. Drug resistant mutations have already been shown to develop in vitro in the presence of HCV protease inhibitors [Lin, et al., “In vitro studies of cross-resistance mutations against two hepatitis C virus serine protease inhibitors, VX-950 and BILN 2061,” J. Biol. Chem. 280, pp. 36784-36791 (2005), which is incorporated herein by reference in its entirety; Lin, et al., “In vitro resistance studies of hepatitis C virus serine protease inhibitors, VX-950 and BILN 2061: Structural analysis indicates different resistance mechanisms,” J. Biol. Chem. 279, pp. 17508-17514 (2004), which is incorporated herein by reference in its entirety; Lu, et al., Antimicrob. Agents Chemother. 48, pp. 2260-6 (2004); Trozzi, et al., “In vitro selection and characterization of hepatitis C virus serine protease variants resistant to an active-site peptide inhibitor,” J. Virol. 77, pp. 3669-79 (2003)]. Mutations resistant to the protease inhibitor BILN 2061 have been found at positions R155Q, A156T, and D168V/AIY in the NS3 gene, but no mutations have yet been observed in the NS4 region or in the protease cleavage sites. A VX-950 resistance mutation has also been found in vitro at position A156S. Cross-resistant mutations against both VX-950 and BILN 2061 have also been shown to develop in vitro at position 156 (A156V/T) (Lin, et al., 2005, supra).

Dosing regimens for VX-950 are described in PCT Publication Number WO 2006/050250, which is incorporated herein by reference in its entirety. Additional dosing regimens for VX-950 are described in PCT Serial Number PCT/US2008/006572, filed on May 21, 2008, which is incorporated herein by reference in its entirety.

### SUMMARY OF THE INVENTION

The invention relates to combination therapies for the treatment of HCV with telaprevir, an oral inhibitor of HCV protease, with pegylated interferon alfa-2a and/or ribavirin. The invention relates to the'treatment of Latino and African American patients infected with HCV using the combination therapy.

In one embodiment, the invention provides a therapeutic regimen comprising administering to a patient pegylated interferon alfa-2a, ribavirin and VX-950, wherein VX-950 is administered in an amount of 750 mg every eight hours, pegylated interferon alfa-2a is administered in an amount of 180 μg per week and ribavirin is administered in an amount of 1000 to 1200 mg per day.

In one embodiment, the invention provides a therapeutic regimen comprising administering to a Latino patient pegylated interferon alfa-2a, ribavirin and VX-950, wherein VX-950 is administered in an amount of 750 mg every eight hours, pegylated interferon alfa-2a is administered in an amount of 180 μg per week and ribavirin is administered in an amount of 1000 to 1200 mg per day.

In one embodiment, the invention provides a therapeutic regimen comprising administering to an African American patient pegylated interferon alfa-2a, ribavirin and VX-950, wherein VX-950 is administered in an amount of 750 mg every eight hours, pegylated interferon alfa-2a is administered in an amount of 180 μg per week and ribavirin is administered in an amount of 1000 to 1200 mg per day.

In one embodiment, the invention provides a therapeutic regimen comprising administering to a patient pegylated interferon alfa-2a, ribavirin and VX-950 in an initial phase and administering pegylated interferon alfa-2a and ribavirin over a secondary phase, wherein the secondary phase occurs after the initial phase and VX-950 is administered in an amount of 750 mg every eight hours, pegylated interferon alfa-2a is administered in an amount of 180 μg per week and ribavirin is administered in an amount of 1000 to 1200 mg per day.

In one embodiment, the invention provides a therapeutic regimen comprising administering to a Latino patient pegylated interferon alfa-2a, ribavirin and VX-950 in an initial phase and administering pegylated interferon alfa-2a and ribavirin over a secondary phase, wherein the secondary phase occurs after the initial phase.

In one embodiment, the invention provides a therapeutic regimen comprising administering to an African American patient pegylated interferon alfa-2a, ribavirin and VX-950 in an initial phase and administering pegylated interferon alfa-2a and ribavirin over a secondary phase, wherein the secondary phase occurs after the initial phase.

In one embodiment, the invention provides a therapeutic regimen comprising administering to a Latino patient pegylated interferon alfa-2a, ribavirin and VX-950 in an initial phase and administering pegylated interferon alfa-2a and ribavirin over a secondary phase, wherein the secondary phase occurs after the initial phase and extends for a period of less than or about 36 weeks.

In one embodiment, the invention provides a therapeutic regimen comprising administering to an African American patient pegylated interferon alfa-2a, ribavirin and VX-950 in an initial phase and administering pegylated interferon alfa-2a and ribavirin over a secondary phase, wherein the secondary phase occurs after the initial phase and extends for a period of less than or about 36 weeks.

The invention includes a diagnostic method useful for determining the dosage level of telaprevir and pegylated interferon alfa-2a necessary to reduce viral breakthrough. The method includes monitoring the blood level of interferon in a patient receiving telaprevir and interferon within the first 12 weeks of therapy; and determining whether to increase the dosage of interferon based upon the level measured blood level of interferon. In one aspect, the blood level of interferon is compared to a predetermined desired blood level of interferon, which can be greater than 5 micrograms/mL, greater than 10 micrograms/mL, greater than 15 micrograms/mL or greater than 20 micrograms/mL. In some aspects the predetermined desired blood level of interferon can be between about 5 to about 15 micrograms/mL.

The invention also includes a method for determining the dosage of telaprevir and interferon necessary to reduce the risk of viral breakthrough. The method includes selecting a desired dose of telaprevir; and determining the minimal dose of interferon which reduces the risk of viral breakthrough. The step of determining the minimal dose of interferon which reduces the risk of viral breakthrough includes comparing the dose of telaprevir with a calibrated plot of viral breakthrough as a function of concentration of telaprevir and interferon.

The invention also includes a method for determining the dosage of telaprevir and interferon necessary to reduce the risk of viral breakthrough. The method includes selecting a desired dose of interferon; and determining the minimal dose of telaprevir which reduces the risk of viral breakthrough. The step of determining the minimal dose of telaprevir which reduces the risk of viral breakthrough includes comparing the dose of interferon with a calibrated plot of viral breakthrough as a function of concentration of telaprevir and interferon.

The addition of telaprevir to the current regimen of Peg-IFN and RBV led to increased SVR rates in clinical trials. 41% of patients achieved SVR with 48 weeks of Peg-IFN/RBV alone, 67% with TVR and 48 weeks Peg-IFN/RBV (p=0.001 versus Peg-IFN/RBV alone), 61% with TVR and 24 weeks Peg IFN/RBV (p=0.02), and 35% with 12 weeks TVR and Peg-IFN/RBV.

Applicants unexpectedly found that telaprevir-based regimens lead to improved viral responses in African Americans and Latinos as compared to Peg-IFN and RBV therapy alone.

### BRIEF DESCRIPTION OF THE FIGURES

FIG. 1 depicts SVR and RVR rates for the PROVE 1 study by race.

FIG. 2 depicts the viral dynamics for the PROVE 1 study during the first 4 weeks of therapy. (A) Compared with Caucasians, Latinos and African Americans have reduced early viral dynamics on Peg-IFN alfa-2a and RBV. (B) On TVR-based treatment, early viral dynamics were more similar among the different racial/ethnic groups.

FIG. 3 depicts the mean hemoglobin levels during the first 12 weeks of therapy in the PROVE 1 study. Mean hemoglobin levels declined with treatment over time with the PR (A) and T/PR (B) regimens. There were no apparent differences between races in mean hemoglobin levels. AA=African American; L=Latino; C=Caucasian.

FIG. 4 depicts the mean absolute neutrophil count during the first 12 weeks of therapy in the PROVE 1 study. Mean absolute neutrophil counts declined with treatment over time with the PR (A) and T/PR (B) regimens. There were no apparent differences between races in mean absolute neutrophil counts. AA=African American; L=Latino; C=Caucasian.

FIG. 5 depicts the PROVE1 study design.

FIG. 6 depicts the PROVE 2 study design.

FIG. 7 depicts the undetectable HCV RNA at Week 4, Week 12 and SVR for the PROVE 2 study. Results were analyzed using the two-sided Fisher's exact test.

FIG. 8 depicts PROVE2 relapse rates 24 Weeks after completion of assigned treatment. Data shown are number of patients with relapse/number of patients with undetectable HCV RNA (<10 IU/mL) at the end of assigned treatment period who met viral response criteria.

FIG. 9 depicts patients with virologic breakthrough at Week 12 for PROVE 2 patients receiving T12/P12, with no RBV.

FIG. 10 depicts patients with virologic breakthrough at Week 12 for PROVE 2 patients receiving T12/PR12 and T12/PR24 combined.

FIG. 11 depicts median hemoglobin levels during the assigned treatment period for the PROVE 2 study. The results show no incremental effect on neutrophil or platelet counts with TVR-based treatment.

### DETAILED DESCRIPTION OF THE INVENTION

VX-950 is described in PCT Publication Numbers WO 02/018369 and WO 2006/050250, and PCT Serial Number PCT/US2008/006572, filed on May 21, 2008, with reference to the following structural formula, or a pharmaceutically acceptable salt thereof:

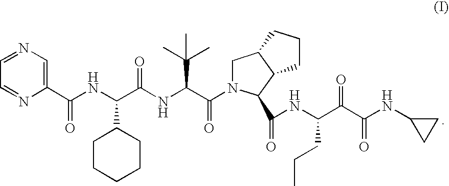

Other descriptions of VX-950 can be found in PCT Publication Numbers WO 07/098,270 and WO 08/106,151.

VX-950 has been tested in single doses in humans and found to be well tolerated (Example 3). The incidence or severity of adverse events did not increase with VX-950 dose. No adverse events were considered to be severe (grade 3 or grade 4). The more common and severe adverse events were skin adverse events (e.g., rash and pruritus), followed by gastrointestinal events and anemia. There were no clinically significant changes from baseline laboratory values for hematology or clinical chemistry parameters. There were no clinically significant changes in physical examinations, vital signs, or electrocardiograms for any subject tested.

Applicants discovered that wild-type HCV may be eradicated by VX-950 within 10 weeks. As to VX-950-resistant variants of HCV (with a 7-20 fold increase in IC₅₀), they may be eradicated by a follow-up of Peg-IFN/RBV dose regimen for 10-24 weeks.

Liver exposures to VX-950 were predicted based on the integrated preclinical and clinical data. The predicted human liver exposures were combined with results of the VX-950 replicon assay and the infectious virus assay to determine the doses that are anticipated to be well tolerated and produce therapeutic benefit. The predicted average liver concentration values are up to 57-fold of the replicon assay IC₉₀ and up to 113-fold of the replicon assay IC₅₀ in the dose range studied.

The results from interim analyses of PROVE 1 and PROVE 2, two large Phase 2b clinical trials evaluating the investigational hepatitis C protease inhibitor telaprevir, dosed in combination with pegylated interferon and ribavirin are described herein. In 24-week telaprevir-based treatment regimens, genotype 1 treatment-naive HCV patients achieved sustained viral response rates of 61% and 65% in PROVE 1 (SVR 12 and SVR 24) and PROVE 2 (SVR 12), respectively. In addition, clinical researchers reported a correlation between achieving rapid viral response (RVR) and achieving SVR in a 24-week telaprevir-based regimen.

Interim analyses of telaprevir safety from PROVE 1 and PROVE 2 appear consistent with prior analyses, with the most common adverse events, regardless of treatment assignment, being fatigue, rash, headache and nausea. Gastrointestinal disorders, skin adverse events (rash, pruritus) and anemia were higher in the telaprevir arms compared to the control arm over the dosing period.

The SVR data from the PROVE studies are promising in that approximately 40% to 50% of people with genotype 1 hepatitis C who undergo 48-week treatment regimens with currently available therapies achieve sustained viral response (SVR). In Phase 2 studied, 24-week telaprevir-based regimens result in SVR of greater than 60% in patients with genotype 1 hepatitis C.

As used herein, “sustained virologic response” or “SVR” is defined as undetectable HCV RNA 24 weeks after end of therapy.

As used herein, the terms “naïve” and “treatment-naïve” refer to a patient who has not receive any prior treatment for Hepatitis C.

According to the present invention “Latino” means any person having origins in any of the original peoples Latin-America or of Spanish-speaking descent.

According to the present invention “African American” means any person having origins in any of the original peoples of Sub-Saharan African ancestry.

Patients normally are requested to self-identify by “race” or the doctor on the basis of their somatic traits and/or the country of origin assigns the race.

In one embodiment, the invention provides a therapeutic regimen comprising administering to a patient pegylated interferon alfa-2a, ribavirin and VX-950, wherein VX-950 is administered in an amount of 750 mg every eight hours, pegylated interferon alfa-2a is administered in an amount of 180 μg per week and ribavirin is administered in an amount of 1000 to 1200 mg per day.

In some embodiments, pegylated interferon alfa-2a, ribavirin and VX-950 are administered in an initial phase and pegylated interferon alfa-2a and ribavirin are administered over a secondary phase, wherein the secondary phase occurs after the initial phase.

In some embodiments, the patient is a treatment naïve patient.

In some embodiments, the patient is a Latino patient.

In some embodiments, the patient is an African American patient.

In some embodiments the secondary phase extends for a period of less than or about 36 weeks and the initial phase extends for a period of less than 24 weeks.

In some embodiments, the initial phase extends for a period of less than 12 weeks.

In some embodiments, the secondary phase extends for a period of less than 24 weeks.

In some embodiments, the secondary phase extends for a period of less than 12 weeks.

In one embodiment, the invention provides a therapeutic regimen comprising administering to a Latino patient pegylated interferon alfa-2a, ribavirin and VX-950, wherein VX-950 is administered in an amount of 750 mg every eight hours, pegylated interferon alfa-2a is administered in an amount of 180 μg per week and ribavirin is administered in an amount of 1000 to 1200 mg per day.

In one embodiment, the invention provides a therapeutic regimen comprising administering to an African American patient pegylated interferon alfa-2a, ribavirin and VX-950, wherein VX-950 is administered in an amount of 750 mg every eight hours, pegylated interferon alfa-2a is administered in an amount of 180 μg per week and ribavirin is administered in an amount of 1000 to 1200 mg per day.

In one embodiment, the invention provides a therapeutic regimen comprising administering to a patient pegylated interferon alfa-2a, ribavirin and VX-950 in an initial phase and administering pegylated interferon alfa-2a and ribavirin over a secondary phase, wherein the secondary phase occurs after the initial phase and VX-950 is administered in an amount of 750 mg every eight hours, pegylated interferon alfa-2a is administered in an amount of 180 μg per week and ribavirin is administered in an amount of 1000 to 1200 mg per day.

In some embodiment, the secondary phase extends for a period of less than or about 36 weeks and the initial phase extends for a period of less than 24 weeks.

In some embodiments, the initial phase extends for a period of less than 12 weeks.

In some embodiments, the secondary phase extends for a period of less than 24 weeks.

In some embodiments, the secondary phase extends for a period of less than 12 weeks.

In one embodiment, the invention provides a therapeutic regimen comprising administering to a Latino patient pegylated interferon alfa-2a, ribavirin and VX-950 in an initial phase and administering pegylated interferon alfa-2a and ribavirin over a secondary phase, wherein the secondary phase occurs after the initial phase.

In some embodiment, VX-950 is administered in an amount of 750 mg every eight hours, pegylated interferon alfa-2a is administered in an amount of 180 μg per week and ribavirin is administered in an amount of 1000 to 1200 mg per day.

In some embodiment, the secondary phase extends for a period of less than or about 36 weeks and the initial phase extends for a period of less than 24 weeks.

In some embodiments, the initial phase extends for a period of less than 12 weeks.

In some embodiments, the secondary phase extends for a period of less than 24 weeks.

In some embodiments, the secondary phase extends for a period of less than 12 weeks.

In one embodiment, the invention provides a therapeutic regimen comprising administering to an African American patient pegylated interferon alfa-2a, ribavirin and VX-950 in an initial phase and administering pegylated interferon alfa-2a and ribavirin over a secondary phase, wherein the secondary phase occurs after the initial phase.

In some embodiments, VX-950 is administered in an amount of 750 mg every eight hours, pegylated interferon alfa-2a is administered in an amount of 180 μg per week and ribavirin is administered in an amount of 1000 to 1200 mg per day.

In some embodiment, the secondary phase extends for a period of less than or about 36 weeks and the initial phase extends for a period of less than 24 weeks.

In some embodiments, the initial phase extends for a period of less than 12 weeks.

In some embodiments, the secondary phase extends for a period of less than 24 weeks.

In some embodiments, the secondary phase extends for a period of less than 12 weeks.

In one embodiment, the invention provides a therapeutic regimen comprising administering to a Latino patient pegylated interferon alfa-2a, ribavirin and VX-950 in an initial phase and administering pegylated interferon alfa-2a and ribavirin over a secondary phase, wherein the secondary phase occurs after the initial phase and extends for a period of less than or about 36 weeks.

In some embodiment, the initial phase extends for a period of less than 24 weeks.

In some embodiments, the initial phase extends for a period of less than 12 weeks.

In some embodiments, the secondary phase extends for a period of less than 24 weeks.

In some embodiments, the secondary phase extends for a period of less than 12 weeks.

In one embodiment, the invention provides a therapeutic regimen comprising administering to an African American patient pegylated interferon alfa-2a, ribavirin and VX-950 in an initial phase and administering pegylated interferon alfa-2a and ribavirin over a secondary phase, wherein the secondary phase occurs after the initial phase and extends for a period of less than or about 36 weeks.

In some embodiment, the initial phase extends for a period of less than 24 weeks.

In some embodiments, the initial phase extends for a period of less than 12 weeks.

In some embodiments, the secondary phase extends for a period of less than 24 weeks.

In some embodiments, the secondary phase extends for a period of less than 12 weeks.

In some embodiments, the invention provides a therapeutic regimen wherein a sustained viral response is achieved.

In certain embodiments, a method according to this invention involves the treatment of a patient infected with genotype 1 Hepatitis C virus. Genotype 1 HCV infection is the most difficult strain of HCV to treat and the most prevalent strain in the United States.

In some embodiments, VX-950 is administered daily at about 450 mg or at about 750 mg every 8 hours, or at about 1250 mg every 12 hours.

Another aspect of this invention provides methods for treating or preventing one or more of liver damage, liver inflammation, steatosis, fatty liver, NAFLD, NASH, alcoholic steatosis, and Reye's syndrome in a patient that is either HCV positive or HCV negative.

Also within the scope of this invention are methods for hepatoprotection in a patient that is either HCV positive or negative.

The amounts of VX-950 according to this invention are administered in a single dosage form or in more than one dosage form. If in separate dosage forms, each dosage form is administered about simultaneously. For the avoidance of doubt, for dosing regimens calling for dosing more than once a day, one or more pill or dose may be given at each time per day (e.g., 1 pill, three times per day or 3 pills, three times per day). Most embodiments of this invention will employ at least 2 pills per dose).

As would be realized by skilled practitioners, if a method of this invention is being used to treat a patient prophylactically, and that patient becomes infected with Hepatitis C virus, the method may then treat the infection. Therefore, one embodiment of this invention provides methods for treating or preventing a Hepatitis C infection in a patient.

In addition to treating patients infected with Hepatitis C, the methods of this invention may be used to prevent a patient from becoming infected with Hepatitis C. Accordingly, one embodiment of this invention provides a method for preventing a Hepatitis C virus infection in a patient comprising administering to the patient a composition or dosage form according to this invention.

Methods of this invention may also involve administration of another component comprising an additional agent selected from an immunomodulatory agent; an antiviral agent; an inhibitor of HCV protease (other than VX-950); an inhibitor of another target in the HCV life cycle (other than NS3/4A protease); an inhibitor of internal ribosome entry, a broad-spectrum viral inhibitor; or a cytochrome P-450 inhibitor; or combinations thereof. The additional agent is also selected from an inhibitor of viral cellular entry.

Accordingly, in another embodiment, this invention provides a method comprising administering VX-950 and another anti-viral agent, preferably an anti-HCV agent. Such anti-viral agents include, but are not limited to, immunomodulatory agents, such as α-, β-, and γ-interferons or thymosin, pegylated derivatized interferon-α compounds, and thymosin; other anti-viral agents, such as ribavirin, amantadine, and telbivudine; other inhibitors of hepatitis C proteases (NS2-NS3 inhibitors and NS3-NS4A inhibitors); inhibitors of other targets in the HCV life cycle, including helicase, polymerase, and metalloprotease inhibitors; inhibitors of internal ribosome entry; broad-spectrum viral inhibitors, such as IMPDH inhibitors (e.g., compounds described in U.S. Pat. Nos. 5,807,876, 6,498,178, 6,344,465, and 6,054,472; and PCT publications WO 97/40028, WO 98/40381, and WO 00/56331; and mycophenolic acid and derivatives thereof, and including, but not limited to, VX-497, VX-148, and VX-944); or any of their combinations.

Other agents (e.g., non-immunomodulatory or immunomodulatory compounds) may be used in combination with a compound of this invention include, but are not limited to, those specified in WO 02/18369, which is incorporated herein by reference (see, e.g., page 273, lines 9-22 and page 274, line 4 to page 276, line 11 this disclosure being specifically incorporated herein by reference).

Still other agents include those described in various published U.S. patent applications. These publications provide additional teachings of compounds and methods that could be used in combination with VX-950 in the methods of this invention, particularly for the treatment of hepatitis. It is contemplated that any such methods and compositions may be used in combination with the methods and compositions of the present invention. For brevity, the disclosure the disclosures from those publications is referred to be reference to the publication number but it should be noted that the disclosure of the compounds in particular is specifically incorporated herein by reference. Examples of such publications include U.S. Patent Application Publication Nos.: US 20040058982, US 20050192212, US 20050080005, US 20050062522, US 20050020503, US 20040229818, US 20040229817, US 20040224900, US 20040186125, US 20040171626, US 20040110747, US 20040072788, US 20040067901, US 20030191067, US 20030187018, US 20030186895, US 20030181363, US 20020147160, US 20040082574, US 20050192212, US 20050187192, US 20050187165, US 20050049220, and US 20050222236.

Still other agents include, but are not limited to, Albuferon™ (albumin-Interferon alpha) available from Human Genome Sciences; PEG-INTRON® (peginterferon alfa-2b, available from Schering Corporation, Kenilworth, N.J.); INTRON-A®, (VIRAFERON®, interferon alfa-2b available from Schering Corporation, Kenilworth, N.J.); ribavirin (1-beta-D-ribofuranosyl-1H-1,2,4-triazole-3-carboxamide, available from ICN Pharmaceuticals, Inc., Costa Mesa, Calif.; described in the Merck Index, entry 8365, Twelfth Edition); REBETROL® (Schering Corporation, Kenilworth, N.J.); COPEGUS® (Hoffmann-La Roche, Nutley, N.J.); PEGASYS® (peginterferon alfa-2a available Hoffmann-La Roche, Nutley, N.J.); ROFERON® (recombinant interferon alfa-2a available from Hoffmann-La Roche, Nutley, N.J.); BEREFOR® (interferon alfa 2 available from Boehringer Ingelheim Pharmaceutical, Inc., Ridgefield, Conn.); SUMIFERON® (a purified blend of natural alpha interferons such as Sumiferon available from Sumitomo, Japan); WELLFERON® (interferon alpha n1 available from Glaxo Wellcome Ltd., Great Britain); ALFERON® (a mixture of natural alpha interferons made by Interferon Sciences, and available from Purdue Frederick Co., CT); α-interferon; natural alpha interferon 2a; natural alpha interferon 2b; pegylated alpha interferon 2a or 2b; consensus alpha interferon (Amgen, Inc., Newbury Park, Calif.); REBETRON® (Schering Plough, Interferon-alpha 2B+Ribavirin); pegylated interferon alpha (Reddy, K. R. et al., “Efficacy and Safety of Pegylated (40-kd) Interferon alpha-2a Compared with Interferon alpha-2a in Noncirrhotic Patients with Chronic Hepatitis C,” Hepatology, 33, 433-438 (2001); consensus interferon (INFERGEN®)(Kao, J. H., et al, “Efficacy of Consensus Interferon in the Treatment of Chronic Hepatitis,” J. Gastroenterol. Hepatol., 15, 1418-1423 (2000); lymphoblastoid or “natural” interferon; interferon tau (C₁₋ayette, P. et al., “IFN-tau, A New Interferon Type I with Antiretroviral activity” Pathol. Biol. (Paris) 47, 553-559 (1999); interleukin-2 (Davis, G. L. et al., “Future Options for the Management of Hepatitis C.” Seminars in Liver Disease, 19, 103-112 (1999); Interleukin-6 (Davis et al., “Future Options for the Management of Hepatitis C,” Seminars in Liver Disease, 19, 103-112 (1999); interleukin-12 (Davis, G. L. et al., “Future Options for the Management of Hepatitis C.” Seminars in Liver Disease, 19, 103-112 (1999); and compounds that enhance the development of type 1 helper T cell response (Davis et al., “Future Options for the Management of Hepatitis C,” Seminars in Liver Disease, 19, 103-112 (1999)). Also included are compounds that stimulate the synthesis of interferon in cells (Tazulakhova, E. B. et al., “Russian Experience in Screening, analysis, and Clinical Application of Novel Interferon Inducers” J. Interferon Cytokine Res., 21 65-73) including, but are not limited to, double stranded RNA, alone or in combination with tobramycin, and Imiquimod (3M Pharmaceuticals; Sauder, D. N. “Immunomodulatory and Pharmacologic Properties of Imiquimod,” J. Am. Acad. Dermatol., 43 S6-11 (2000). See also, WO 02/18369, particularly page 272, line 15 to page 273, line 8, this disclosure being specifically incorporated herein by reference.

As is recognized by skilled practitioners, VX-950 is preferably administered orally. Interferon is not typically administered orally, although orally administered forms are in development. Nevertheless, nothing herein limits the methods or combinations of this invention to any specific dosage forms or regime. Thus, each component of a combination according to this invention may be administered separately, together, or in any combination thereof. As recognized by skilled practitioners, dosages of interferon are typically measured in IU (e.g., about 4 million IU to about 12 million IU). Interferon may also be dosed by micrograms. For example, a standard dose of Peg-Intron is 1.0-1.5 μg/kg/wk and of Pegasys is 180 μg/wk.

In some aspects, the method includes the administration of agents over two phases, an initial phase and a secondary phase. For instance the initial phase can be a period of less than about 12 or 24 weeks and the secondary phase can be greater or equal to about 12 weeks, e.g., the secondary phase can be between about 12-36 weeks. In certain embodiments, the secondary phase is 12 weeks. In still other embodiments, the secondary phase is 36 weeks. In certain embodiments, the sum of the initial and secondary phase is about 24 to 48 weeks (such as 24, 36, or 48 weeks). In some embodiments, the initial and secondary phases can be identical in duration.

VX-950 may be administered in either the initial, secondary, or both phases. In some embodiments, VX-950 is administered only in the initial phase. When VX-950 is administered only in the initial phase, VX-950 may be administered alone or in combination with other agents and one or more agents are administered in the secondary phase. The other agents can be one or more anti-viral agents, one or more other agents described herein, or combinations thereof. In some embodiments, the specific agents administered in the initial and secondary phases are identical.

In some embodiments, the method includes the administration of VX-950 for 12 weeks (initial phase) followed by 12 weeks of administration of a combination of Peginterferon alfa-2a (Peg-IFN) and ribavirin (RBV) (secondary phase). In other embodiments, the method includes the administration of VX-950 for 12 weeks (initial phase) followed by 24 weeks of administration of a combination of Peg-IFN and RBV (secondary phase). In other embodiments, the method includes the administration of VX-950 for 12 weeks (initial phase) followed by 36 weeks of administration of a combination of Peg-IFN and RBV (secondary phase).

In still other embodiments, the method includes the administration of VX-950 for 12 weeks in combination with Peg-IFN (initial phase) followed by 12 weeks of administration of a combination of Peg-IFN and RBV (secondary phase). In other embodiments, the method includes the administration of VX-950 for 12 weeks in combination with Peg-IFN (initial phase) followed by 24 weeks of administration of a combination of Peg-IFN and RBV (secondary phase). In other embodiments, the method includes the administration of VX-950 for 12 weeks in combination with Peg-IFN (initial phase) followed by 36 weeks of administration of a combination of Peg-IFN and RBV (secondary phase).

In still other embodiments, the method includes the administration of VX-950 for 12 weeks in combination with Peg-IFN and RBV (initial phase) followed by 12 weeks of administration of a combination of Peg-IFN and RBV (secondary phase). In other embodiments, the method includes the administration of VX-950 for 12 weeks in combination with Peg-IFN and RBV (initial phase) followed by 24 weeks of administration of a combination of Peg-IFN and RBV (secondary phase). In other embodiments, the method includes the administration of VX-950 for 12 weeks in combination with Peg-IFN and RBV (initial phase) followed by 36 weeks of administration of a combination of Peg-IFN and RBV (secondary phase).

In some embodiments, any of the initial phases described above can be conducted for about 12 weeks and the secondary phases can be conducted for about 12 weeks. Alternatively, the initial phase can be conducted for about 12 weeks and the secondary phase can be conducted for about 24 weeks. In still other aspects, the initial phase can be conducted for about 12 weeks and the secondary phase can be conducted for about 36 weeks.

In some embodiments, any of the initial phases described above can be conducted for about 8 weeks and the secondary phases can be conducted for about 16 weeks. Alternatively, the initial phase can be conducted for about 8 weeks and the secondary phase can be conducted for about 28 weeks. In still other aspects, the initial phase can be conducted for about 8 weeks and the secondary phase can be conducted for about 40 weeks.

In some embodiments, the method includes administering VX-950 in combination with Peg-IFN for less than 48 weeks. For instance, the method includes administering VX-950 in combination with Peg-IFN for less than 24 weeks.

In some embodiments, the method includes administering VX-950 in combination with Peg-IFN and RBV for less than 48 weeks. For instance, the method includes administering VX-950 in combination with Peg-IFN and RBV for less than 24 weeks.

Modeling data also indicate that VX-950 resistant variants, such as V36A/M, T54A, R155K/T, A156S A156V/T, V36A/M-R155K/T, and V36A/M-A156V/T, may be eradicated mainly by administering PEG-IFN and ribavirin for about 10-24 weeks (or 8-26 weeks) following VX-950 treatment. Certain of these regimens represent a reduction in treatment in the current standard of care treatment regimen lasting 24-48 weeks.

In some embodiments, the method of this invention is able to achieve week 4 RVR and week 12 undetectable status.

Accordingly, this invention also provides methods for administering VX-950 in combination with an interferon. In certain embodiments, the interferon is administered for about 10 weeks (or 10 weeks), about 12 weeks (or 12 weeks), about 14 weeks (or 14 weeks). Ribavirin is also optionally administered for all or part of the regimen, including but not limited to, the entire regimen.

In one embodiment, a method of this invention comprises administering a combination of VX-950 and Peg-IFN for about 12 weeks (or 12 weeks).

In one embodiment, a method of this invention comprises administering a combination of VX-950 and Peg-IFN for about 12±4 weeks (e.g., 8, 12, or 16 weeks).

In one embodiment, a method of this invention comprises administering a combination of VX-950 and Peg-IFN for about 24 weeks (or 24 weeks).

In one embodiment, a method of this invention comprises administering a combination of VX-950 and Peg-IFN for about 24±4 weeks (e.g., 20, 24, or 28 weeks).

For the avoidance of doubt, it should be understood that this invention includes, but is not limited to, a regimen involving administering VX-950 and an interferon for about 8 weeks (or 8 weeks) followed by administering interferon for about 16 weeks (or 16 weeks) for a total treatment regimen of about 24 weeks (or 24 weeks). Also provided is a regimen involving administering VX-950 and an interferon for about 12 weeks (or 12 weeks) followed by administering interferon for about 12 weeks (or 12 weeks) for a total treatment regimen of about 24 weeks (or 24 weeks). Such regimens optionally provide administration of ribavirin for all or part of the regimen, including but not limited to, the entire regimen of about 24 weeks (or 24 weeks).

In one embodiment, a method of this invention comprises administering a combination of VX-950, Peg-IFN, and ribavirin for about 12 weeks (or 12 weeks).

In one embodiment, a method of this invention comprises administering a combination of VX-950, Peg-IFN, and ribavirin for about 12 weeks (or 12 weeks) followed by administering Peg-IFN and ribavirin for about 12 weeks (or 12 weeks).

In one embodiment, a method of this invention comprises administering a combination of VX-950, Peg-IFN, and ribavirin for about 12 weeks (or 12 weeks) followed by administering Peg-IFN and ribavirin for about 36 weeks (or 36 weeks).

In one embodiment, a method of this invention comprises administering a combination of VX-950, Peg-IFN, and ribavirin for about 24 weeks (or 24 weeks) followed by administering Peg-IFN and ribavirin for about 24 weeks (or 24 weeks).

In some embodiments, the method includes providing a loading dose of VX-950 (1250 mg) followed by 750 mg q8h VX-950 plus a combination of Peg-IFN and RBV.

A cytochrome P450 monooxygenase (“CYP”) inhibitor used in connection with this invention is expected to inhibit metabolism of VX-950. Therefore, the cytochrome P450 monooxygenase inhibitor would be in an amount effective to inhibit metabolism of VX-950. Accordingly, the CYP inhibitor is administered in an amount such that the bioavailability of or exposure to VX-950 is increased in comparison to VX-950 in the absence of the CYP inhibitor. CYP inhibitors include, but are not limited to, ritonavir (WO 94/14436), ketoconazole, troleandomycin, 4-methylpyrazole, cyclosporin, clomethiazole, cimetidine, itraconazole, fluconazole, miconazole, fluvoxamine, fluoxetine, nefazodone, sertraline, indinavir, nelfinavir, amprenavir, fosamprenavir, saquinavir, lopinavir, delavirdine, erythromycin, VX-944, and VX-497. Preferred CYP inhibitors include ritonavir, ketoconazole, troleandomycin, 4-methylpyrazole, cyclosporin, and clomethiazole.

Methods for measuring the ability of a compound to inhibit cytochrome P50 monooxygenase activity are known (see, U.S. Pat. No. 6,037,157, and Yun et al., Drug Metabolism & Disposition, 21, 403-407 (1993)). Methods for evaluating the influence of co-administration of VX-950 and a CYP inhibitor in a subject are also known (US 2004/0028755). Any such methods could be used in connection with this invention to determine the pharmacokinetic impact of a combination.

One embodiment of this invention provides a method for administering an inhibitor of CYP3A4 and VX-950.

The methods herein may involve administration or co-administration of a) combinations of VX-950 and another agent; or b) VX-950 in more than one dosage form. Co-administration includes administering each inhibitor in the same dosage form or in different dosage forms. When administered in different dosage forms, the inhibitors may be administered at different times, including about simultaneously or in any time period around administration of the other dosage forms. Separate dosage forms may be administered in any order. That is, any dosage forms may be administered prior to, together with, or following the other dosage forms.

VX-950, and any additional agent, may be formulated in separate dosage forms. Alternatively, to decrease the number of dosage forms administered to a patient, VX-950, and any additional agent, may be formulated together in any combination. Any separate dosage forms may be administered at the same time or different times. It should be understood that dosage forms should be administered within a time period such that the biological effects were advantageous.

According to the regimens and dosage forms of this invention, VX-950 is present in an amount effective to decrease the viral load in a sample or in a patient, wherein said virus encodes a NS3/4A serine protease necessary for the viral life cycle (or in an amount effective to carry out a method of this invention), and a pharmaceutically acceptable carrier. Alternatively, a composition of this invention comprises an additional agent as described herein. Each component may be present in individual compositions, combination compositions, or in a single composition.

If pharmaceutically acceptable salts of compounds are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentane-propionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate. Base salts include ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

Also, the basic nitrogen-containing groups may be quaternized with such agents as lower alkyl halides, such as methyl, ethyl, propyl, and butyl chloride, bromides and iodides; dialkyl sulfates, such as dimethyl, diethyl, dibutyl and diamyl sulfates, long chain halides such as decyl, lauryl, myristyl and stearyl chlorides, bromides and iodides, aralkyl halides, such as benzyl and phenethyl bromides and others. Water or oil-soluble or dispersible products are thereby obtained.

The compounds utilized in the compositions and methods of this invention may also be modified by appending appropriate functionalities to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

According to a preferred embodiment, the compositions of this invention are formulated for pharmaceutical administration to a mammal, particularly a human being.

Formulations of VX-950 are described in PCT Publication Numbers WO 05/123076, WO 07/109,604 and WO 07/109,605, which are incorporated herein by reference in their entirety.

Such pharmaceutical compositions of the present invention (as well as compositions for use in methods, combinations, kits, and packs of this inventions) may be administered orally, parenterally, sublingually, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally or intravenously. More preferably, the compositions are administered orally.

Sterile injectable forms of the compositions of and according to this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In compositions of this invention comprising VX-950 and an additional agent, VX-950 and the additional agent should be present at dosage levels of between about 10 to 100%, and more preferably between about 10 to 80% of the dosage normally administered in a monotherapy regi men.

The pharmaceutical compositions of this invention may be orally administered in any orally acceptable dosage form including, but not limited to, capsules, tablets, pills, powders, granules, aqueous suspensions or solutions. In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. Acceptable liquid dosage forms include emulsions, solutions, suspensions, syrups, and elixirs.

Alternatively, the pharmaceutical compositions of this invention may be administered in the form of suppositories for rectal administration. These may be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

As is recognized in the art, pharmaceutical compositions may also be administered in the form of liposomes.

Applicants have demonstrated that VX-950 is orally bioavailable. Accordingly, preferred pharmaceutical compositions of this invention are formulated for oral administration.

For the CYP inhibitor, the dosage levels of between about 0.001 to about 200 mg/kg body weight per day, would be typical. More typical would be dosage levels of between about 0.1 to about 50 mg/kg or about 1.1 to about 25 mg/kg per day.

For preferred dosage forms of ritonavir, see U.S. Pat. No. 6,037,157, and the documents cited therein: U.S. Pat. No. 5,484,801, U.S. patent application Ser. No. 08/402,690, and PCT Publications Nos. WO 95/07696 and WO 95/09614.

Administrations in connection with this invention can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Preferably, such preparations contain from about 20% to about 80% active compound.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. Patients may, however, require intermittent treatment on a long-term basis upon any recurrence of disease symptoms.

It should also be understood that a specific dosage and treatment regimen for any particular patient will depend upon a variety of factors, including the activity of the specific compound employed, the age, body weight, general health, sex, diet, time of administration, rate of excretion, drug combination, the judgment of the treating physician and the severity of the particular disease being treated, prior treatment history, co-morbidities or concomitant medications, baseline viral load, race, duration of diseases, status of liver function and degree of liver fibrosis/cirrhosis, and the goal of therapy (eliminating circulating virus per-transplant or viral eradication). The amount of active ingredients will also depend upon the particular described compound and the presence or absence and the nature of the additional anti-viral agent in the composition.

According to another embodiment, the invention provides a method for treating a patient infected with a virus characterized by a virally encoded NS3/4A serine protease that is necessary for the life cycle of the virus by administering to said patient a pharmaceutically acceptable composition of this invention. Preferably, the methods of this invention are used to treat a patient suffering from a HCV infection. Such treatment may completely eradicate the viral infection or reduce the severity thereof. Preferably, the patient is a mammal. More preferably, the patient is a human being.

The dosages herein are preferably for use in vivo. Nevertheless, this is not intended as a limitation to using of these amounts of VX-950 for any purpose. In yet another embodiment the present invention provides a method of pre-treating a biological substance intended for administration to a patient comprising the step of contacting said biological substance with a pharmaceutically acceptable composition comprising a compound of this invention. Such biological substances include, but are not limited to, blood and components thereof such as plasma, platelets, subpopulations of blood cells and the like; organs such as kidney, liver, heart, lung, etc; sperm and ova; bone marrow and components thereof, and other fluids to be infused into a patient such as saline, dextrose, etc.

This invention also provides a process for preparing a composition comprising VX-950, or a pharmaceutically acceptable salt thereof, and a pharmaceutically acceptable carrier, adjuvant; or vehicle comprising the step of combining the VX-950, or the pharmaceutically acceptable salt thereof, and the pharmaceutically acceptable carrier, adjuvant, or vehicle, wherein the dosage of VX-950 in the composition is in accordance with any embodiment of this invention. An alternative embodiment of this invention provides a process wherein the composition comprises one or more additional agent as described herein.

This invention also provides a therapeutic regimen comprising VX-950, or a pharmaceutically acceptable salt thereof, at the dosages disclosed herein. In an alternative embodiment of this invention, the therapeutic regimen further comprises one or more of additional agent as described herein.

Pharmaceutical compositions may also be prescribed to the patient in “patient packs” containing the whole course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in traditional prescriptions. The inclusion of a package insert has been shown to improve patient compliance with the physician's instructions.

It will be understood that the administration of the combination of the invention by means of a single patient pack, or patient packs of each formulation, containing within a package insert instructing the patient to the correct use of the invention is a desirable additional feature of this invention.

According to a further aspect of the invention is a pack including VX-950 (in dosages according to this invention) and an information insert containing directions on the use of the combination of the invention. Any composition, dosage form, therapeutic regimen or other embodiment of this invention may be presented in a pharmaceutical pack. In an alternative embodiment of this invention, the pharmaceutical pack further comprises one or more of additional agent as described herein. The additional agent or agents may be provided in the same pack or in separate packs.

Another aspect of this involves a packaged kit for a patient to use in the treatment of HCV infection or in the prevention of HCV infection (or for use in another method of this invention), comprising: a single or a plurality of pharmaceutical formulation of each pharmaceutical component; a container housing the pharmaceutical formulation(s) during storage and prior to administration; and instructions for carrying out drug administration in a manner effective to treat or prevent HCV infection.

Accordingly, this invention provides kits for the simultaneous or sequential administration of a dose of VX-950 (and optionally an additional agent). Typically, such a kit will comprise, e.g. a composition of each compound and optional additional agent(s) in a pharmaceutically acceptable carrier (and in one or in a plurality of pharmaceutical formulations) and written instructions for the simultaneous or sequential administration.

In another embodiment, a packaged kit is provided that contains one or more dosage forms for self administration; a container means, preferably sealed, for housing the dosage forms during storage and prior to use; and instructions for a patient to carry out drug administration. The instructions will typically be written instructions on a package insert, a label, and/or on other components of the kit, and the dosage form or forms are as described herein. Each dosage form may be individually housed, as in a sheet of a metal foil-plastic laminate with each dosage form isolated from the others in individual cells or bubbles, or the dosage forms may be housed in a single container, as in a plastic bottle. The present kits will also typically include means for packaging the individual kit components, i.e., the dosage forms, the container means, and the written instructions for use. Such packaging means may take the form of a cardboard or paper box, a plastic or foil pouch, etc.

A kit according to this invention could embody any aspect of this invention such as any composition, dosage form, therapeutic regimen, or pharmaceutical pack.

The packs and kits according to this invention optionally comprise a plurality of compositions or dosage forms. Accordingly, included within this invention would be packs and kits containing one composition or more than one composition.

Although certain exemplary embodiments are depicted and described below, it will be appreciated that compounds of this invention can be prepared according to the methods described generally above using appropriate starting materials generally available to one of ordinary skill in the art.

All cited documents are incorporated herein by reference.

In order that this invention be more fully understood, the following preparative and testing examples are set forth. These examples are for the purpose of illustration only and are not to be construed as limiting the scope of the invention in any way.

### EXAMPLES

### Example 1

### PROVE 1 and PROVE 2 Clinical Studies

PROVE 1 is an ongoing, four-arm, Phase 2b clinical trial of 250 treatment-naive genotype 1 HCV patients with a primary objective to assess the proportion of patients who achieve SVR, defined as undetectable (less than 10 IU/mL, as measured by the Roche TaqMan(R) assay) HCV RNA 24 weeks after the completion of dosing. The trial is assessing patients who receive telaprevir-based treatment regimens of 12, 24 and 48 week durations, compared to a 48-week control arm of pegylated-interferon and ribavirin. PROVE 1 is being conducted at more than 30 clinical centers in the U.S.

Baseline patient characteristics were similar across telaprevir treatment and control arms in PROVE 1. Twenty percent of those treated with telaprevir were either Hispanic (10%) or African American (10%). In the control arm, 8% of patients were Hispanic and 12% were African American. Median HCV RNA at entry was similar across all arms (6.6 Log 10 IU/mL in telaprevir treatment arms and 6.7 Log 10IU/mL in control) and 87% of patients had a high viral load, defined as >800,000 IU/mL. On average, patients were 49 years old (21-63 years range) with a mean weight of 82.1 kg (46-136 kg range).

PROVE 2 is an ongoing, four-arm, Phase 2b clinical trial of 323 treatment-naive genotype 1 HCV patients with a primary objective to assess the proportion of patients who achieve SVR. The study is assessing patients who receive telaprevir-based treatment regimens of 12, 24 and 48 week durations, compared to a 48-week control arm. PROVE 2 is being conducted at more than 40 clinical centers in Europe.

The median baseline viral load for patients in PROVE 2 was 6.4 Log 10 IU/mL (3.3-7.7) and 83% of patients had a high viral load, defined as >800,000 IU/mL. The majority of patients were male (94.1%), Caucasian (94.1%) and infected with genotype 1b (54.1%) compared to genotype 1a (34.1%). On average, patients were 45 years old (18-65 years range) with a mean weight of 70.9 kg (45-115 kg range).

Sustained viral responses (SVR) across PROVE 1 and PROVE 2 are outlined in the Table 1 below,

TABLE 1
Sustained Viral Response for PROVE 1 and PROVE 2 studies.

| Treatment Arm (Study) | ITT^{a} | SVR Rate |
|---|---|---|
| 24-week treatment arm | n = 79 | 61% |
| (PROVE 1) | | |
| 24-week treatment arm | n = 81 | 65%^{b} |
| (PROVE 2) | | |
| 12-week treatment arm with ribavirin (PROVE 1) | n = 17 | 35% |
| 12-week treatment arm with ribavirin (PROVE 2) | n = 82 | 59% |

^{a}ITT = Intention-to-treat; missing = failure
^{b}SVR12: undetectable HCV RNA <10 IU/mL at 12 weeks post-treatment and is an interim measurement. Other data represent SVR 24, defined as undetectable HCV RNA <10 IU/mL at 24 weeks post-treatment. Across all the treatment arms above, there were no relapses between 12 and 24 weeks follow-up, i.e. there was 100% concordance between SVR 12 and SVR 24.
In addition, the SVR rate in the 12-week arm without ribavirin (n = 78) in PROVE 2 was 29%.

In the 48-week telaprevir treatment arm (12+36; n=79) of PROVE 1, 65% had undetectable HCV RNA (<10 IU/mL) at end of treatment.

Sustained viral response results from the control arms of PROVE 1 and PROVE 2 are not available. At the time of the interim analysis, in the PROVE I control arm (n=75), 45% of patients receiving 48-weeks of pegylated interferon and ribavirin had undetectable HCV RNA (<10 IU/mL) at end of treatment. At the time of the interim analysis, in the control arm of PROVE 2 (n=82), 59% of patients receiving 48 weeks of peg-IFN and RBV had undetectable HCV RNA (<10 IU/mL) at week 36 on-treatment. Typically, following the completion of 48 weeks of treatment with peg-IFN+RBV, a certain proportion of patients with undetectable HCV RNA relapse.

SVR rates given for the telaprevir arms include patients who completed dosing in their study arm as well as patients who discontinued treatment prior to completion of dosing, but who met the criteria for SVR 24 (defined as undetectable HCV RNA <10 IU/mL 24 weeks after completing treatment).

In PROVE 1 and PROVE 2 combined, on an ITT basis, 77% of patients receiving telaprevir in combination with peg-IFN and RBV achieved a rapid viral response at 4 weeks (79% in PROVE 1, 75% in PROVE 2), defined as undetectable HCV RNA <10 IU/mL as measured by the Roche TagMan(R) assay, compared to an average of 12% of patients across the control arms of PROVE 1 and PROVE 2 (11% in PROVE 1, 13% in PROVE 2; p<0.001 for the comparison in each study).

For those patients that achieved RVR, completed 24 weeks of telaprevir-based therapy, and had data available for SVR analysis, 91% achieved an SVR 24 or SVR 12. This finding demonstrates a correlation between RVR and SVR in a 24-week telaprevir-based treatment regimen.

In PROVE 1 and PROVE 2 combined, 5% of patients receiving telaprevir in combination with peg-IFN and RBV experienced viral breakthrough in the first 12 weeks of treatment (7% in PROVE 1, 2% in PROVE 2). Most viral breakthroughs occurred in the first month of treatment, and were generally associated with low interferon blood levels. After patients had undetectable HCV RNA (<10 TU/mL), less than 2% of patients receiving telaprevir in combination with peg-IFN and RBV experienced viral breakthrough on treatment.

In PROVE 1 and PROVE 2 combined, the relapse rate for patients who completed 24 weeks of treatment was 9% (2% in PROVE 1, 14% in PROVE 2). In PROVE 1 and PROVE 2 combined, for those patients that achieved an RVR and completed 24 weeks of therapy, 7% experienced viral relapse in the post-treatment period (2% in PROVE 1, 11% in PROVE 2). Per protocol in PROVE 1, only patients who achieved an RVR were to stop treatment at 24 weeks of therapy; no such criteria were utilized in PROVE 2. Following completion of treatment, no patient in PROVE 1 that received telaprevir in combination with peg-IFN and RBV relapsed after week 12 of the 24-week post-treatment period.

The types of adverse events that have been commonly observed with Peg-IFN and RBV were seen across all treatment arms of PROVE 1 and PROVE 2. The most common adverse events, regardless of treatment assignment, were fatigue, rash, headache and nausea. Gastrointestinal disorders, skin adverse events (rash, pruritus) and anemia were higher in the telaprevir arms compared to the control arm over the dosing period.

In PROVE 1, the overall discontinuation rate through 12 weeks was 18% across all telaprevir treatment arms and 3% in the control arm. This includes discontinuations due to adverse events, withdrawal of consent and patients lost to follow-up. The incidence of treatment discontinuations through week 12 due to adverse events was 13% and 2% in the telaprevir and control arms, respectively. The most common reason for discontinuation was rash, with 7% of patients discontinued for this reason in the telaprevir arms during the first 12 weeks of treatment. After week 12, discontinuations due to adverse events were 8% each in the telaprevir and control arms. Over the full course of the treatment period, the incidence of severe adverse events was 27% in the telaprevir arms and 24% in the control arm.

In PROVE 2, the overall discontinuation rate through 12 weeks was 14% across all telaprevir treatment arms and 6% in the control arm. This includes discontinuations due to adverse events, withdrawal of consent and patients lost to follow-up. The incidence of treatment discontinuations through week 12 due to adverse events were 10% and 3% in the telaprevir and control arms, respectively. As with PROVE 1, the most common reason for discontinuation was rash, with 7% of patients discontinued due to rash in the telaprevir arms, compared to less than one percent in the control arm during the first 12 weeks of treatment. Through to week 12, the time of the interim safety analysis being reported, the incidence of severe adverse events was 17% in the telaprevir arms and 10% in the control arm.

### Example 2

### Tolerance and Pharmacokinetics Studies

VX-950 was examined in a randomized, double-blind, placebo-controlled single-dose escalation study. 25 healthy male volunteers were enrolled and each received multiple single doses of VX-950 (at least 7 days apart, 3 doses of VX-950 at increasing dose levels) and 1 dose of placebo.

Doses of 25 mg to 1250 mg were evaluated. A dose escalation scheme was used that combined dose doubling and modified Fibonacci to be aggressive in the lower dose range and conservative in the higher dose range.

The results showed that VX-950 was well tolerated at all dose levels. No serious adverse events were reported during the study, and there did not appear to be an increase in adverse events with increasing dose levels.

### Example 3

### Viral Responses in African-Americans, Latinos and Caucasians

African Americans and Latinos have much lower sustained virologic response (SVR) rates to current treatment for chronic hepatitis C virus (HCV) compared to Caucasians. A sub-analysis of African Americans (AA), Latinos (L) and Caucasians (C) shows that the addition of telaprevir to the peginterferon-alfa and ribavirin (PR) treatment leads to increased SVR rates in the PROVE 1 trial.

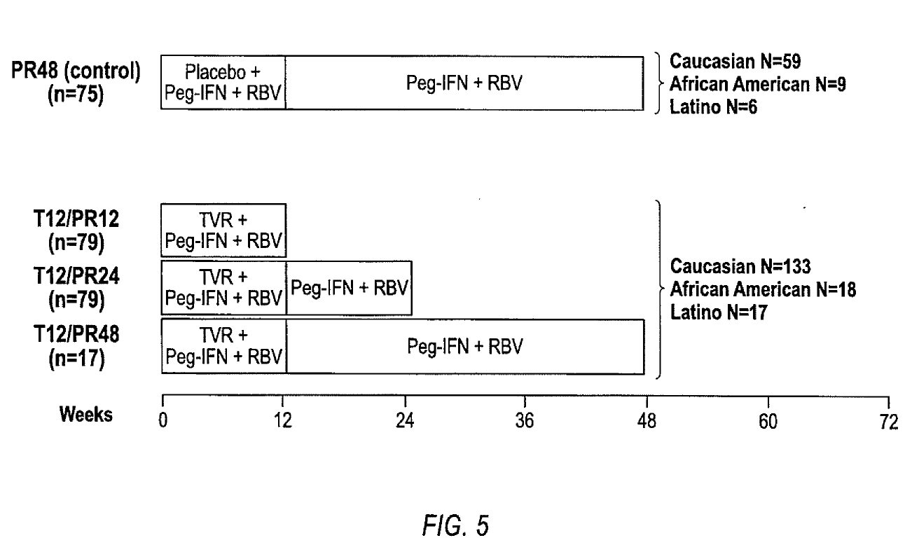

In the study, patients received TVR 750 mg Oh with peginterferon alfa 2a 180 μg/week and ribavirinI000-1200 mg/day, in naive subjects with genotype 1 HCV infection. Subjects were randomized into 4 arms (FIG. 5). The control arm (n=75) received 48 weeks of PR (PR arm). The 3 other arms all received TVR for 12 wks in combination with 12, 24 or 48 wks of PR (T/PR arm, n=175). This analysis focuses on the viral responses and pharmacokinetics of African American, Latino and Caucasian subjects in these arms. Race and ethnicity were determined by subject self-reporting.

The Roche COBAS TagMan® assay was used to measure HCV RNA (LOD 10 IU/mL). For viral kinetic modeling, values reported as <10 IU/mL were replaced with 5 IU/mL.

As shown in Table 2, baseline characteristics were well balanced across groups. Enrollment of Caucasians (73.8%, n=192) was greater than African Americans (10.4%, n=27) and Latinos (8.8%, n=23).

TABLE 2
Baseline characteristics of PROVE 1.

| | PR48 (n = 75) | | | | | | T/PR arms (n = 168) | | | | | |
|---|---|---|---|---|---|---|---|---|---|---|---|---|
| | | | African | | | | | | African | | | |
| | Caucasian | | American | | Latino | | Caucasian | | American | | Latino | |
| | N = 59 | | N = 9 | | N = 6 | | N = 133 | | N = 18 | | N = 17 | |
| Male, n (%) | 37 | (63) | 3 | (33) | 2 | (33) | 90 | (68) | 10 | (56) | 11 | (65) |
| Age, median | 49 | (27-59) | 50 | (28-58) | 44 | (24-56) | 50 | (21-63) | 50 | (31-57) | 48 | (32-60) |
| (range) | | | | | | | | | | | | |
| BMI, median | 26.6 | (20-37) | 31.6 | (19-38) | 27.6 | (23-37) | 25.8 | (18-43) | 33.2 | (20-44) | 28.7 | (21-37) |
| (range) | | | | | | | | | | | | |
| Mean HCV | 6.74 | (0.47) | 6.46 | (0.47) | 6.34 | (0.48) | 6.51 | (0.64) | 6.58 | (0.72) | 6.46 | (0.49) |
| RNA log10 | | | | | | | | | | | | |
| IU/mL (SD) | | | | | | | | | | | | |
| >800k IU/mL, | 56 | (95) | 8 | (89) | 4 | (67) | 114 | (86) | 15 | (83) | 15 | (88) |
| n (%) | | | | | | | | | | | | |
| HCV genotype, | | | | | | | | | | | | |
| n (%) | | | | | | | | | | | | |
| 1a | 39 | (66) | 6 | (67) | 4 | (67) | 87 | (65) | 10 | (56) | 10 | (59) |
| 1b | 16 | (27) | 2 | (22) | 2 | (33) | 36 | (27) | 7 | (39) | 5 | (29) |
| 1 (other) | 4 | (7) | 1 | (11) | 0 | (0) | 10 | (8) | 1 | (6) | 2 | (12) |
| Bridging | 13 | (22) | 3 | (33) | 3 | (50) | 20 | (15) | 4 | (22) | 7 | (41) |
| fibrosis, n (%) | | | | | | | | | | | | |

In the PR arm, the difference in viral decline at wk 1 is significantly different between the Caucasian and African American subgroups (p=0.04); in the T/PR groups, there is no significant difference between these subgroups (p=−0.36) [P values could not be calculated for the Latino group because of the small number]. As shown in Table 3, SVR rates among subjects receiving TVR appear increased compared to the PR arm in Caucasian (82/133, 62% vs. 27/59, 46%), African American (8/18, 44% vs. 1/9, 11%) and Latino (11/17, 65% vs. 2/6, 33%) subjects. For the African Americans that achieved SVR the distribution among treatment arms is as follows: T12/PR12, n=3; T12/PR24, n=1; and T12/PR48, n=4. For the Latinos that achieved SVR the distribution among treatment arms is as follows: T12/PR12, n=1; T12/PR24, n=6; and T12/PR48, n=4.

TABLE 3
Viral responses throughout treatment and follow-up.

| | | | | |
|---|---|---|---|---|
| Wk1 | | | % with | |
| HCV | | % with | cEVR | |
| RNA | | EVR 2- | undetect- | |
| log10 | % with | log drop at | able at | % with |
| decline | RVR | Week 12 | Week 12 | SVR |

| | PR | T/PR | PR | T/PR | PR | T/PR | PR | T/PR | PR | T/PR |
|---|---|---|---|---|---|---|---|---|---|---|
| C | −1.3 | −4.7 | 12 | 81 | 81 | 77 | 49 | 73 | 46 | 62 |
| AA | −0.6 | −4.4 | 11 | 78 | 44 | 72 | 22 | 68 | 11 | 44 |
| L | −0.6 | −4.3 | 0 | 65 | 83 | 88 | 33 | 82 | 33 | 65 |

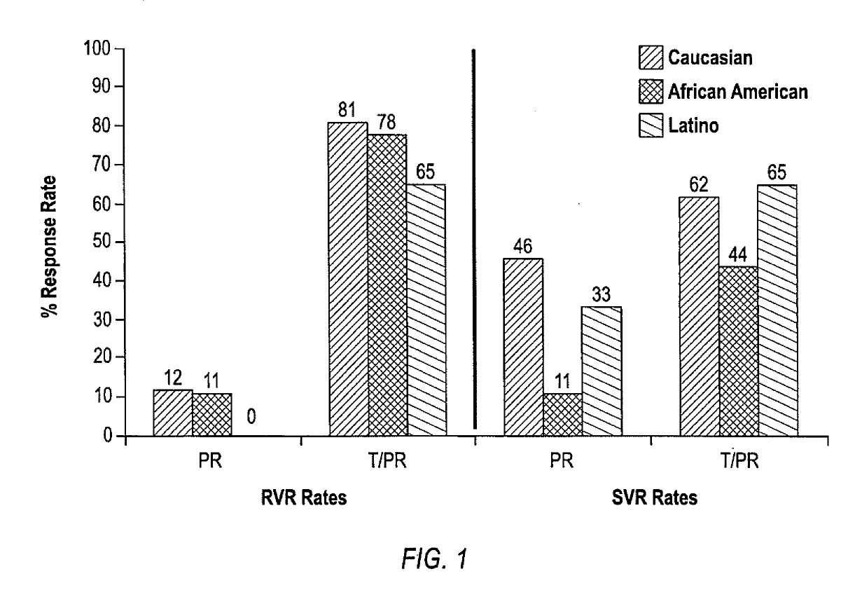

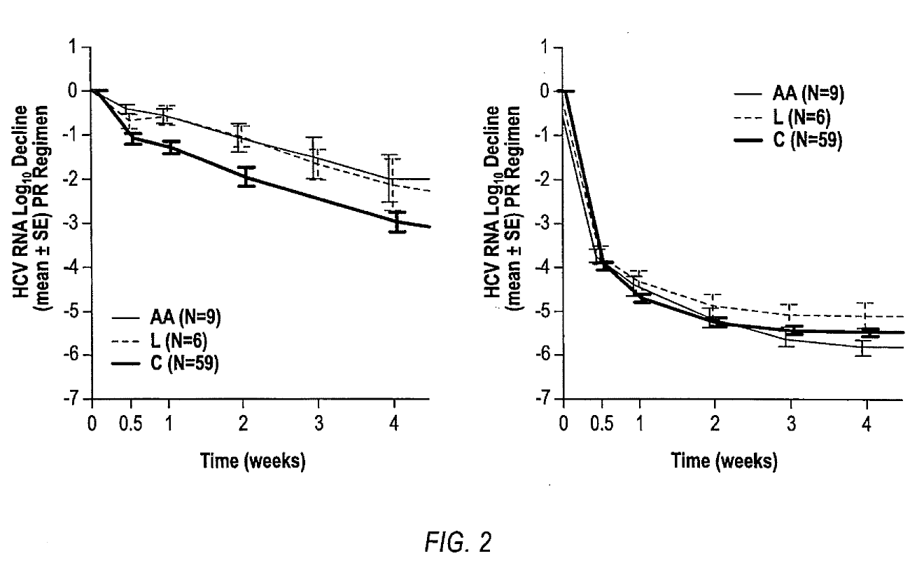

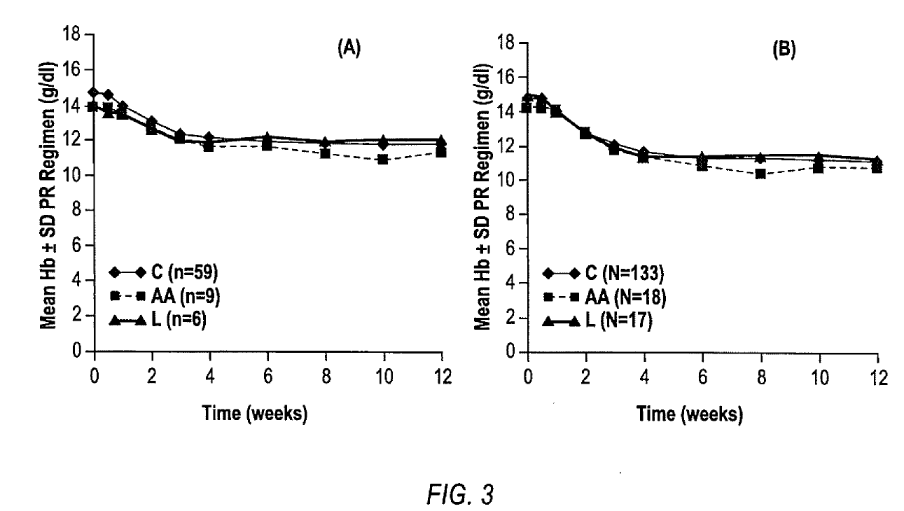

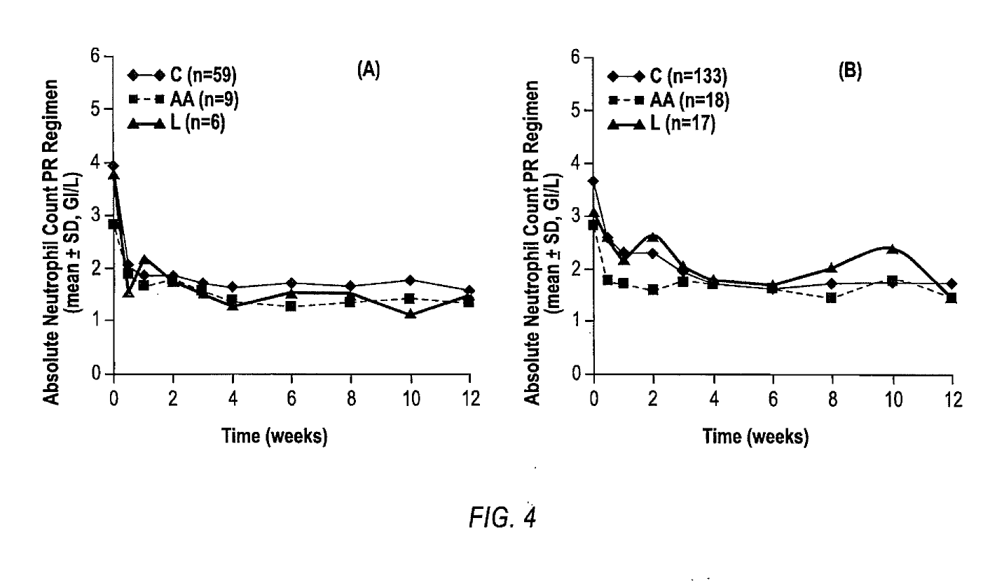

Telaprevir-based regimens enhance early viral kinetics and subsequently lead to improved viral responses in African Americans, Latinos and Caucasians (FIG. 1). FIG. 2 shows the viral dynamics during the first 4 weeks of therapy. Panel A demonstrates that, compared with Caucasians, Latinos and African Americans have reduced early viral dynamics on Peg-IFN and RBV; Panel B reveals that with the addition of TVR to Peg-IFN alfa-2a and RBV, improved early viral dynamics were observed for all groups and were similar among the different racial/ethnic groups. No differences were observed in the pharmacokinetics of telaprevir among the different racial/ethnic groups (FIGS. 3 and 4).

In the overall study population, the most common adverse events (AEs) reported more frequently than placebo were gastrointestinal events, skin events (rash, pruritus) and anemia. Treatment discontinuations through week 12 due to skin/rash AEs were 7% in the T/PR arms and 1% in the PR arm.

Table 4 summarizes the more common adverse events in the different groups. Adverse events were included in the table if the rate was greater than 20% in a treatment group or, if a group had less than 10 subjects, at least 3 subjects in the group experienced the adverse event. There were no apparent differences in adverse event profiles in the different racial/ethic groups, given the small group sizes. No rashes described as moderate or severe were reported in African American and Latino subjects.

TABLE 4
Adverse Events to Week 48, Report in >20% of Subjects in Any Racial/ethnic
Group.*

| | | | African | | | |
|---|---|---|---|---|---|---|
| | Caucasians | | Americans | | Latinos | |
| Adverse event, | PR | All T/PR | PR | All T/PR | PR | All T/PR |
| n (%) | n = 59 | n = 133 | n = 9 | n = 18 | n = 6 | n = 17 |
| Fatigue | 47 (80) | 103 (77) | 5 (56) | 6 (33) | 4 (67) | 13 (77) |
| Influenza-like illness | 28 (48) | 55 (41) | 0 | 4 (22) | 3 (50) | 11 (65) |
| Injection Site Erythema | 16 (27) | 44 (33) | 2 (22) | 3 (17) | 0 | 4 (24) |
| Fever | 17 (29) | 25 (19) | 3 (33) | 1 (6) | 2 (33) | 4 (24) |
| Irritability | 20 (34) | 18 (14) | 0 | 3 (17) | 1 (17) | 1 (6) |
| Chills | 12 (20) | 24 (18) | 1 (11) | 3 (17) | 1 (17) | 1 (6) |
| Pain | 14 (24) | 11 (8) | 1 (11) | 2 (11) | 0 | 2 (12) |
| Nausea | 20 (34) | 74 (56) | 1 (11) | 7 (39) | 1 (17) | 6 (35) |
| Diarrhea | 19 (32) | 54 (41) | 1 (11) | 5 (28) | 1 (17) | 2 (12) |
| Vomiting | 6 (10) | 29 (22) | 2 (22) | 4 (22) | 1 (17) | 3 (18) |
| Rash (any type - mild) | 18 (32) | 49 (37) | 1 (11) | 6 (33) | 3 (50) | 8 (47) |
| Rash (any type - | 5 (8) | 26 (20) | 1 (11) | 0 | 0 | 0 |
| moderate) | | | | | | |
| Rash (any type - | 1 (2) | 12 (9) | 0 | 0 | 0 | 0 |
| severe) | | | | | | |
| Pruritus | 16 (27) | 52 (39) | 0 | 7 (39) | 1 (17) | 12 (71) |
| Dry skin | 16 (27) | 25 (19) | 2 (22) | 1 (6) | 0 | 2 (12) |
| Generalized Pruritus | 0 | 18 (14) | 0 | 5 (28) | 0 | 1 (6) |
| Headache | 34 (57) | 60 (45) | 6 (67) | 5 (28) | 4 (67) | 10 (59) |
| Dizziness | 11 (19) | 32 (24) | 1 (11) | 3 (17) | 1 (17) | 3 (18) |
| Insomnia | 25 (42) | 58 (44) | 2 (22) | 5 (28) | 2 (33) | 3 (18) |
| Depression | 12 (20) | 28 (21) | 1 (11) | 2 (11) | 2 (33) | 3 (18) |
| Cough | 13 (22) | 28 (21) | 0 | 2 (11) | 1 (17) | 3 (18) |
| Arthralgia | 12 (20) | 27 (20) | 2 (22) | 3 (17) | 1 (17) | 2 (12) |
| Myalgia | 12 (20) | 20 (15) | 5 (56) | 5 (28) | 1 (17) | 1 (6) |
| Anemia | 16 (27) | 44 (33) | 1 (11) | 5 (28) | 2 (33) | 7 (41) |
| Neutropenia | 13 (22) | 23 (17) | 3 (33) | 2 (11) | 1 (17) | 2 (12) |
| Anorexia | 2 (3) | 2 (2) | 2 (22) | 1 (6) | 0 | 0 |

*Or in ≧3 subjects if n < 10 in a group.

In some embodiments of the invention, dosing regimens for treating African Americans, Latinos and Caucasians include those described in WO 2006/050250. Additional dosing regimens for VX-950 are described in PCT Serial Number PCT/US2008/006572, filed on May 21, 2008, which is incorporated herein by reference in its entirety.

### Example 4

### Telaprevir in Combination with Peginterferon-Alfa-2a with or without Ribavirin

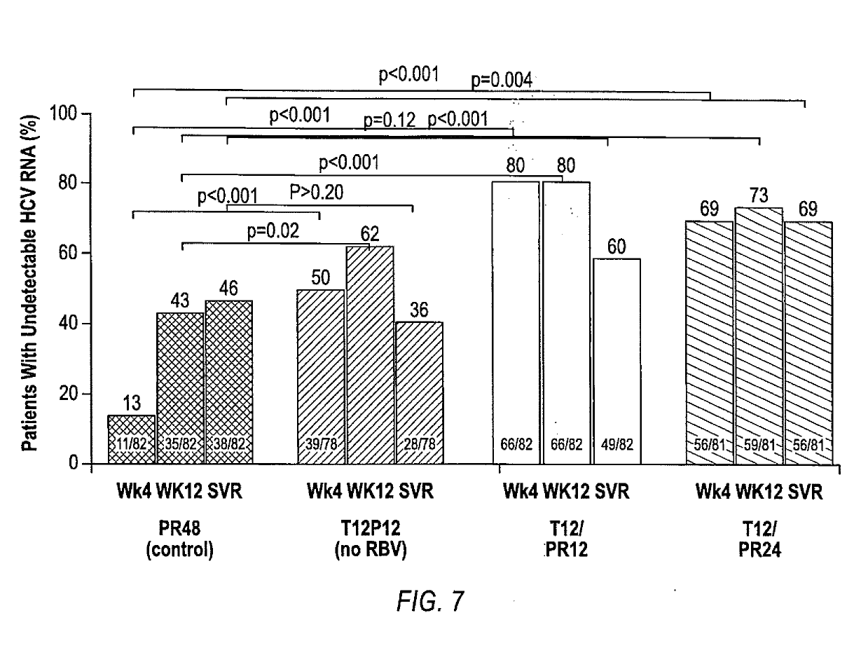

Telaprevir produces rapid and consistent reductions of HCV RNA plasma levels (FIG. 7). The PROVE 2 trial was designed to assess safety and efficacy of TVR in combination with Peg-IFN alfa-2a with or without ribavirin in chronic HCV genotype-1 treatment-naïve patients without cirrhosis.

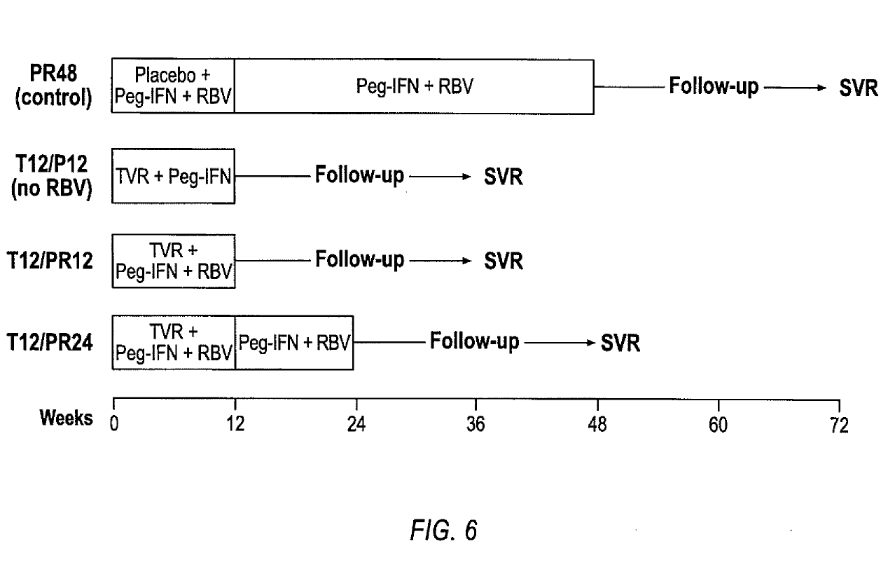

In the PROVE 2 study, 323 patients were randomized to (i) Peg-IFN 180 μg/wk, RBV 1000 or 1200 mg/day, TVR-placebo 48 wks (PR48; n=82); (ii) TVR 750 mg q8h, Peg-IFN+RBV for 12 wks, then Peg-IFN+RBV for 12 wks (T12/PR24; n=81); (iii) TVR 750 mg q8h, Peg-IFN+RBV 12 wks (T12/P12; n=82); or (iv) TVR+Peg-IFN 12 wks (T12/P12; n=78) (FIG. 6). The primary endpoint was sustained virologic response (undetectable HCV RNA 24 weeks after end of therapy).

As shown in Table 5, baseline characteristics were well balanced across groups. The overall population was 59% male; 94% Caucasian; Age: 45 (range 18-65); BMI 23.75 kg/m² (range 17-41); HCV RNA 6.5 log₁₀ IU/mL (3.4-7.7), 86% HCV RNA >600,000 IU/mL, genotype 1a/1b: 44%155%, ALT: 55; 7% METAVIR F3. Rapid and sustained virologic response, as well as relapse rates and adverse effects (AE) leading to discontinuation are given in Table 6.

TABLE 5
Baseline characteristics of PROVE 2.

| | PR48 | T12/P12 | | |
|---|---|---|---|---|
| | (control) | (no RBV) | T12/PR12 | T12/PR24 |
| | (n = 82) | (n = 78) | (n = 82) | (n = 81) |
| Male, n (%) | 46 (56) | 43 (55) | 49 (60) | 54 (67) |
| Caucasian, n (%) | 76 (93) | 77 (99) | 76 (93) | 75 (93) |
| Age, | 45 (18-64) | 45 (20-64) | 44 (22-65) | 46 (19-65) |
| median (range) | | | | |
| BMI, | 24 (17-35) | 24 (18-41) | 23 (17-32) | 24 (17-35) |
| median (range) | | | | |
| ALT (U/L), | 55 (20-315) | 58 (18-303) | 50 (15-259) | 56 (18-277) |
| median (range) | | | | |
| METAVIR F3, | 5 (6) | 4 (5) | 4 (5) | 12 (15) |
| n (%) | | | | |
| Mean HCV RNA | 6.5 (4.8-7.4) | 6.4 (3.9-7.3) | 6.5 (3.4-7.3) | 6.6 (4.1-7.6) |
| log₁₀ IU/mL | | | | |
| (range) | | | | |
| HCV RNA ≧800k | 68 (83) | 63 (81) | 65 (79) | 72 (89) |
| IU/mL, n (%) | | | | |
| HCV | | | | |
| genotype, n (%) | | | | |
| 1a | 35 (43) | 40 (51) | 37 (45) | 31 (38) |
| 1b | 45 (55) | 38 (49) | 45 (55) | 50 (62) |
| 1 (subtype | 2 (2) | 0 | 0 | 0 |
| unknown) | | | | |

METAVIR F3 = historical evidence of bridging fibrosis; Genotype by NS3-4A sequencing analysis.

TABLE 6
Results of the PROVE 2 study.

| Endpoint | PR48 | T12/P12 | T12/PR12 | T12/PR24 |
|---|---|---|---|---|
| RVR (%) | 13 | 51 | 80 | 69 |
| SVR rate (%) | 48 | 36 | 62 | 68 |
| Relapse rates (%) | 20 | 48 | 29 | 14 |
| AEs leading to | 10 | 10 | 12 | 16 |
| discontinuation through wk | | | | |
| 48 (% pts) | | | | |

PR48 12-wk follow-up post-treatment in this table;
RVR = rapid virologic response;
AE = adverse event.
p values [2-sided Fisher's exact test] vs PR48 for SVR: T12/P12 p > 0.20;
T12/PR12 p = 0.08;
T12/PR24 p = 0.01.

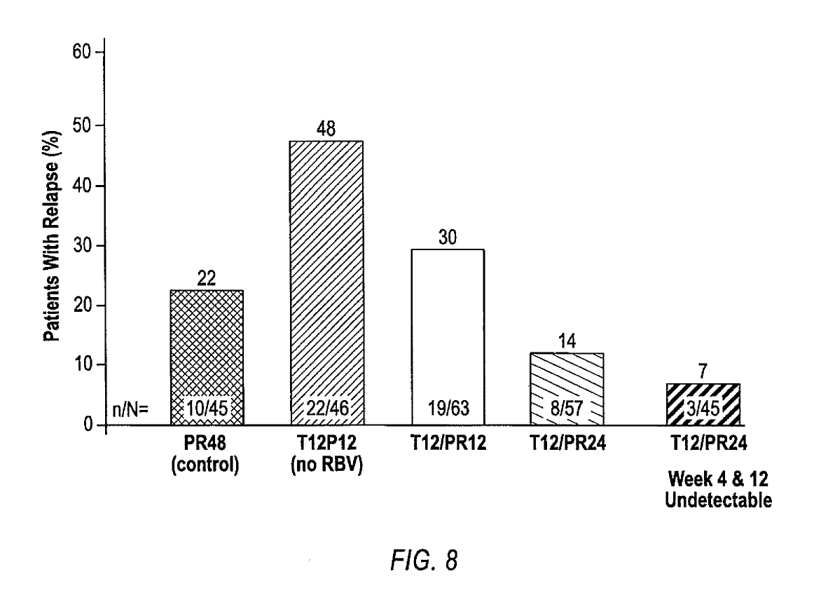

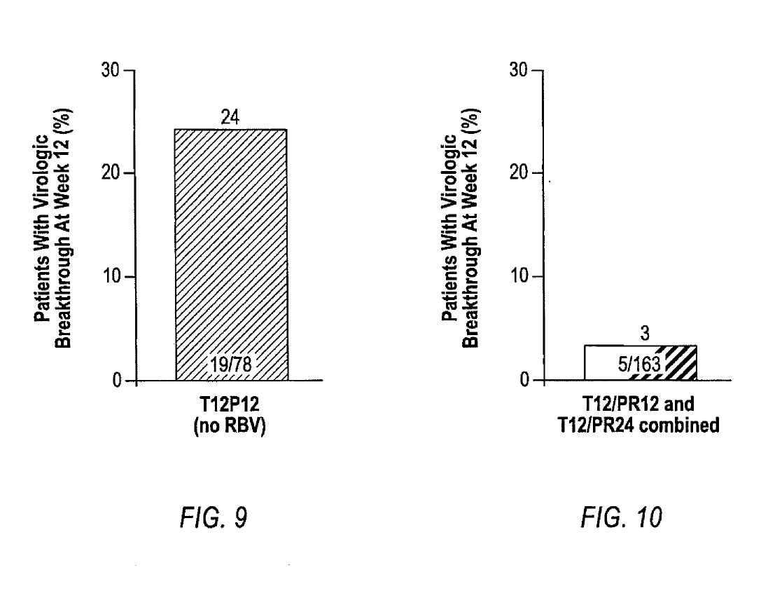

As shown in FIG. 8, relapse rate in patients receiving T12/PR24 with 4-wk and 12-wk undetectable HCV RNA was 7% (3/45). Virologic breakthrough (at wk 12 in patients on treatment; >1 log₁₀ increase from nadir or (100 IU/mL HCV RNA after prior undetectable) was 24% (T12/P12) (FIG. 9) and 3% (T12/PR12 and T12/PR24 combined) (FIG. 10), suggesting that ribavirin is still a critical component of the regimen. In patients with virologic breakthrough on the T12/P12 regimen, the following NS3 variants were detected: V36M/R155K, R155K, A156T, A156S, T54T/A, T54A/A156S, V36V/A, A156S/T, T54T/A, R155R/K, and A156A/S/T. In patients with virologic breakthrough on the T12/PR12 and T12/PR24 regimens combined, the following NS3 variants were detected: V36M/R155K and A156T.

As shown in Table 7, the most common AEs through wk 48 reported in ≧25% of patients regardless of severity in any treatment arm. AEs included pruritus, rash, anemia, fatigue, weakness and headaches. Most AEs were grade 1 or 2. Grade 3 AEs in (3% pts were reported for PR48: anemia (4%); T12/P12: rash (3%) and depression (3%); T12/P12: rash (6%) and asthenia/fatigue (5%); T12/PR24: rash (7%). Table 8 shows the reasons for discontinuation in all treatment arms.

TABLE 7
Common adverse invents for PROVE 2.

| | PR48 | T12/P12 | | |
|---|---|---|---|---|
| | (control) | (no RBV) | T12/PR12 | T12/PR24 |
| Adverse Event | (n = 82) | (n = 78) | (n = 82) | (n = 81) |
| Pruritus | 35 | 59 | 63 | 51 |
| Rash, all types | 35 | 47 | 44 | 49 |
| Nausea | 40 | 31 | 48 | 48 |
| Asthenia | 32 | 38 | 52 | 46 |
| Headache | 45 | 47 | 39 | 44 |
| Influenza-like | 52 | 36 | 39 | 40 |
| illness | | | | |
| Insomnia | 39 | 14 | 34 | 28 |
| Anemia | 17 | 9 | 18 | 27 |
| Dry skin | 35 | 28 | 26 | 26 |
| Diarrhea | 28 | 26 | 32 | 25 |
| Fatigue | 37 | 33 | 28 | 26 |
| Dyspnea | 16 | 14 | 26 | 22 |
| Cough | 26 | 10 | 17 | 19 |
| Arthralgia | 17 | 26 | 10 | 10 |

TABLE 8
Safety observations, discontinuations.

| | PR48 | T12/P12 | | |
|---|---|---|---|---|
| | (control) | (no RBV) | T12/PR12 | T12/PR24 |
| Discontinuations n (%) | (n = 82) | (n = 78) | (n = 82) | (n = 81) |
| All | 16 (19) | 8 (10) | 10 (12) | 20 (25) |
| Adverse event n, % | 6 (7) | 7 (9) | 9 (11) | 11 (14) |
| Lost to Follow-up | 2 (2) | 0 | 0 | 0 |
| Others* | 8 (10) | 1 (1) | 1 (1) | 9 (6) |
| Most common AEs | | | | |
| resulting in | | | | |
| treatment | | | | |
| discontinuation: | | | | |
| Rash, all types | 0 | 2 (3) | 6 (7) | 6 (7) |
| Gastrointestinal | 1 (1) | 0 | 1 (1) | 1 (1) |
| Pruritus | 0 | 2 (3) | 1 (1) | 1 (1) |
| Anemia | 1 (1) | 2 (3) | 1 (1) | 1 (1) |

*Others includes the following causes: termination of the study by investigator discretion, withdrawal of consent, non-compliance, refusal of treatment, non-responder, enrollment error.

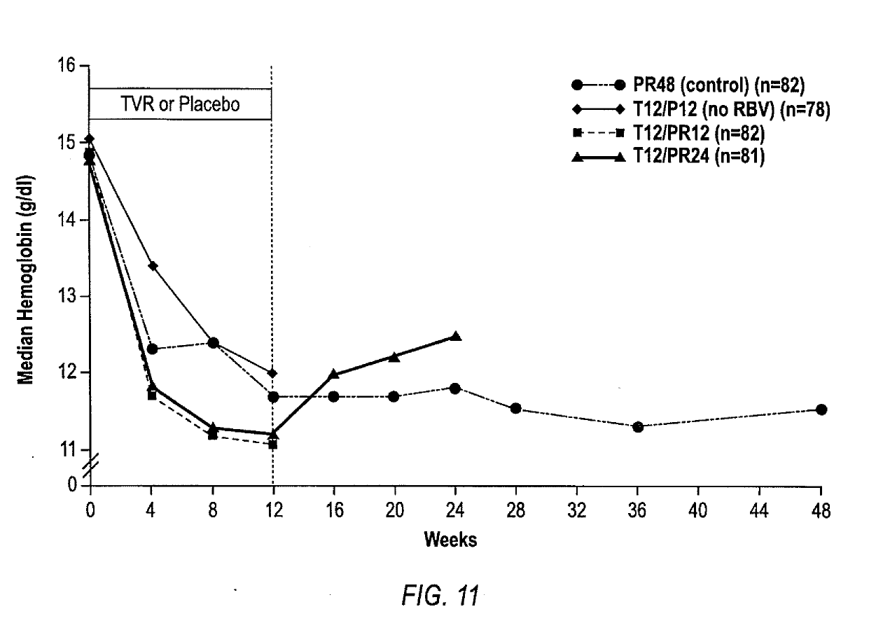

Treatment with TVR-based regimens did not result in additional neutropenia or thrombocytopenia. FIG. 11 shows the median hemoglobin levels during the assigned treatment period for each arm of the study.

RVR rates for African Americans and Caucasians were similar (72% versus 80%) in the T/PR arms. The discrepancy between the high RVR rate and the lower SVR rate for African Americans was largely related to treatment discontinuation. RVR and SVR rates for Latinos were similar to Caucasians.

Telaprevir in combination with Peg-IFN/RBV demonstrated significantly higher SVR rates compared with the control group in patients infected with HCV genotype 1, with the potential to shorten the overall treatment duration by half in most patients.

TABLE 9
Multivariate analysis for Prove 2.

| Factor | P-value | Odds Ratio (95% CI) |
|---|---|---|
| Prescribed Drug Regimen | | |
| T12/PR24 vs. control | 0.001 | 3.04 (1.55-5.96) |
| T12/PR12 vs. control | 0.09 | 1.75 (0.910-3.35) |
| T12/P12 vs. control | 0.18 | 0.63 (0.32-1.24) |
| Baseline Characteristics | | |
| Genotype subtype: 1b vs. 1a | 0.08 | 1.54 (0.96-2.49) |
| Viral load: <800K vs. ≧800K | <0.001 | 4.41 (2.15-9.05) |
| Age: ≦45 vs. >45 years | 0.06 | 1.58 (0.98-2.56) |

Gender, BMI, ALT and glucose did not have a strong association (P > 0.20) with SVR; CI Confidence Intervals.

### Example 5

The following example details a process of fluidized spray drying (FSD) and provides the results of fluidized spray drying two mixtures, a mixture of HPMCAS polymer and solvents (placebo) and a mixture of VX-950, HPMCAS, and solvents (active). By varying parameters of the FSD process, the properties of the resulting product can be optimized and tailored for subsequent processing or use.

### Objectives

The examples presented herein were designed in part:
i) To describe spray drying studies carried out on a VX-950 dispersion using a commercial spray dryer operating in Fluidized Spray Dryer mode (for example, a dryer with a capacity of 1.250 kg/hr operating in FSD mode)
ii) To report the effect of variations in selected operating parameters on product density, particle size distribution, and residual solvents.

### Introduction

Increased particle size and/or product density are advantageous to obtaining a direct compressible product. A commercial scale spray dryer (for example, a spray dryer with a capacity of 1250 kg/hr) configured as a Fluidized Spray Dryer (FSD mode) to obtain larger particles and product with a suitably high density, e.g., for direct compression, was used. To accomplish a direct compressible material, it is sometimes desirable to increase the average particle size from the range of 20-40 μm to higher levels, while maintaining or increasing product density (e.g., bulk density >0.2 g/ml and tap density >0.4 g/ml). An additional criterion is to be able to reduce the level of residual solvents, after post-drying, to within acceptable limits.

The analytical work on the spray dried material and final product involved the analysis of particle properties (product density and particle size distribution) and the level of residual solvents.

### Equipment, Materials, and Methods

Two feeds were prepared during the current study. The placebo feed for the high drug formula (placebo) and the respective high drug load formula (active). Table 10 summarizes the feeds spray dried in each experiment.

TABLE 10
Correspondence between feeds, batches, formula and amounts
of solids and solvents used.

| | | Feed 1 | Feed 2 |
|---|---|---|---|
| Formula | | placebo | active |
| VX-950 | kg | — | 25 |
| HPMCAS | kg | 80 | 5 |
| TOTAL SOLIDS | kg | 80 | 30 |
| DCM | kg | 1920 | 120 |
| TOTAL SOLVENTS | kg | 1920 | 120 |
| C_feed | % w/w | 4.0 | 20.0 |

Composition of the solid dispersion (% w/w)

| | | |
|---|---|---|
| VX-950 | — | 83.3 |
| HPMCAS | 100 | 16.6 |

Composition of the solvent (% w/w)

| | | |
|---|---|---|
| DCM | 100 | 100 |

The feeds were prepared in an 8000-L stainless steel stir tank reactor equipped with a mechanical stirrer and thermal circuit for controlling the temperature of the feed. During the preparation oithe placebo batch, the solvent was charged to the reactor before charging the polymer (HPMCAS). Complete dissolution was observed under low to moderate stirring (between 30 and 80 rpm). In the active tests, the solids were charged first and thereafter the solvent. Dissolution took about 6 hours. The temperature of the solutions in the feed reactor was kept at about 20° C. (between 15 and 30° C.) while waiting to be fed to the spray drier.

### Fluidized Spray Drying of Placebo Feed and Active Feed

A stainless steel commercial scale spray dryer (NIRO, size 4) equipped with a pressure nozzle atomization system was used in the tests. The atomization nozzle used was from Spraying Systems (MFP (Maximum Free Passage) SK Series SPRAYDRY® Nozzles Series variety, orifice 52 with core 27).

The spray drying unit was operated in closed cycle mode, i.e., with recirculation of the drying gas. The spray drying unit included a supply tank containing a solvent (T510) for use during start-up and shut-down operations, and a supply tank containing the material to be dried (R240). To start the spray drying process, valve V2 was opened and the material to be spray dried was fed from the supply tank R240 to the spray drying chamber DC via pump HP-P. The material was partially dried in the drying chamber and then the lighter dried particles exited to the cyclone C with the drying gas, while the heavier particles fell down into fluidized bed FB1. From FB1, the particles eventually circulated to secondary fluidized beds FB2 and FB3 to complete their cooling and drying. The light particles (fines) that went out to cyclone C were then separated out by the cyclone and returned to the drying chamber at the fines return FR. Any tiny particles that passed through the cyclone were caught by the filter bag FB prior to the gas recycling unit RU.

Recirculation of the drying gas was accomplished by recirculating the gas from the recycling unit through one or the other of the closed loops indicated by flow paths (1) and (2). The path taken by the gas exiting the recycling unit was determined by valving (not shown). The gas was recycled through flow path (2) to carry fines from the cyclone back to the drying chamber DC. The gas was also re-circulated to the drying chamber, as drying gas for the drying chamber DC, through a heat exchanger HX1.

The flow of drying nitrogen, controlled by a set-point in the blowing fan (F1), was adjusted to obtain a pressure drop across the cyclone (AP_syclone) between 10 and 18 cm H₂O. A high pressure pump was used (HP—P), and the feed pressure (P-feed) was controlled automatically by imposing the desired set-point value (P_feed_SP). The fines return position (FR position) was either set to the top of the drying chamber (to promote agglomeration) or to the middle of the drying chamber (to decrease agglomeration). When the valve to closed loop (1) was open, gas was fed to the fluidized chambers FB1-FB3 by an independent fan (VT-FB) and the temperature of each of the three fluidizing chambers (T_FB1, T_FB2, T_FB3) was controlled by three heat-exchangers (HE1, HE2, HE3). These were set to the test values (30, 35, and 40° C., respectively).

The feed was atomized at the nozzle's tip and was dried in the drying chamber by the co-current hot nitrogen. The stream containing the dried product inverted direction within the drying chamber, exiting at the top before entering the cyclone, where most of the solids were separated and the fines were re-introduced into the drying chamber either at the top (to be mixed with the spray formed at the nozzle) or axially to the middle of the drying chamber. As discussed above, the heavier particles formed during drying and/or during the agglomeration process fell down within the drying chamber and into the main fluidizing chamber (FB1). The process proceeded until a given layer of product (measured as a differential pressure across FB1) was obtained. Part of the product in FB1 was then discharged to FB2 where a post-drying process occurred, after which the product in FB2 was transferred to FB3. In FB3 the product was cooled to ambient temperature before final discharge to the packaging room. As discussed above, after leaving the cyclone the nitrogen passed through a filter bag, where finer particles were caught, before entering exhaust fan (F2) and the gas recycling unit from which it was recirculated through loops (1) and/or (2). The exhaust fan speed was adjusted to control the pressure within the system.

### Materials

The materials used during the tests are presented in Table 11.

TABLE 11
Materials used during the spray drying studies.

| Material | Supplier |
|---|---|
| VX-950 | RPS-Annan (manufacturer) |
| HPMCAS | SHIN-ETSU (manufacturer) |
| Dichloromethane | ARAGONESAS (manufacturer) |
| (methylene chloride) | |

### Analytical Methods

The analytical controls applied were bulk and tap density (e.g., measured by United States Pharmacopeia (USP) method <601>), particle size distribution by typical volumetric laser diffraction (e.g., Malvern Mastersizer, or Sympatec HELOS or MYTOS), and organic solvents (dichloromethane (DCM), acetone and ethyl acetate) by gas chromatography (GC).

### Results and Discussion

### Spray Drying Tests: Data and Observations

Seven spray drying tests were carried out (five placebo and two active). The principal results are summarized in Table 12. Scanning Electron Microscope (SEM) pictures were taken. Pictures were taken of dispersions prepared with the fines being introduced at the top of the spray dryer and with the fines being introduced at the middle of the spray dryer. Introducing the fines at the top of the spray dryer yielded a more agglomerated product. Introducing the fines at the middle of the spray dryer yielded a less agglomerated product. Pictures were taken at 30×, 100×, and 300× magnifications.

TABLE 12
Results of fluidized spray drying.

| Test number | | | | | | |
|---|---|---|---|---|---|---|
| 01 | 02 | 03 | 04 | 05 | 06 | 07 |

| | | |
|---|---|---|
| Formula | placebo | active |

Feed properties and spray drying parameters

| | | | | | | | | |
|---|---|---|---|---|---|---|---|---|
| Feed used | kg | 681 | 432 | 205 | 243 | 243 | 88 | 62 |
| C_feed | % w/w | 4.0 | 4.0 | 4.0 | 4.0 | 4.0 | 20.0 | 20.0 |
| Feed viscosity | Cp | 27.2 | 27.2 | 27.2 | 27.2 | 27.2 | N/A | N/A |
| T_in | ° C. | 75 ± 3 | 90 | 85 ± 2 | 71 ± 1 | 70 ± 1 | 75 ± 3 | 75 ± 3 |
| T_out | ° C. | 40 ± 1 | 40 | 40 | 30 ± 1 | 31 ± 3 | 35 ± 5 | 43 ± 2 |
| ΔP cyclone | cm H₂O | 15-18 | 15-18 | 11-13 | 10-14 | 10-13 | 10-12 | 15-18 |
| P_feed_SP | bar | 22 | 40 | 22 | 22 | 22 | 22 | 22 |
| Drying time | min | 210 | 115 | 74 | 91 | 89 | 35 | 25 |
| F_feed | kg/h | 195 | 225 | 166 | 175 | 164 | 151 | 149 |
| FR position | — | Top | Top | Top | Top | Middle | Middle | Middle |
| T_FB1_SP | ° C. | 80 | 90 | 90 | 90 | 90 | 90 | 90 |
| T_FB2_SP | ° C. | 80-90 | 90 | 90 | 90 | 90 | 90 | 90 |
| T_FB3_SP | ° C. | 0 | 0 | 0 | 0 | 0 | 0 | 0 |
| V_FB1, 2, 3 | % open | 25, 25, 50 | 25, 25, 50 | 25, 25, 50 | 25, 25, 50 | 25, 25, 50 | 25, 25, 50 | 25, 25, 50 |
| VT-FB | % | 10 | 10 | 5 | 4 | 4 | 4 | 30 |

Process throughput and yield

| | | | | | | | | |
|---|---|---|---|---|---|---|---|---|
| F_solids^{a)} | kg/h | 7.8 | 9.0 | 6.6 | 7.0 | 6.6 | 30.2 | 29.8 |

| | | | |
|---|---|---|---|
| Yield^{b)} | % w/w | 77 | 135 |

Product properties*

| Sample Number | | 338691 | 338693 | 339695 | 338699 | 338699 | 338702 | 338703 |
|---|---|---|---|---|---|---|---|---|
| Bulk density | g/ml | 0.14 | 0.13 | 0.14 | 0.17 | 0.20 | 0.32 | 0.25 |
| Tap density | g/ml | 0.18 | 0.18 | 0.19 | 0.23 | 0.25 | 0.41 | 0.32 |
| d10 | μm | 123.73 | 116.25 | 106.58 | 129.03 | 94.16 | 16.47 | 13.37 |
| d50 | μm | 238.95 | 245.35 | 225.08 | 258.54 | 186.07 | 60.03 | 51.45 |
| d90 | μm | 413.05 | 456.44 | 419.83 | 487.94 | 338.51 | 151.05 | 141.67 |
| Span | — | 1.21 | 1.39 | 1.39 | 1.39 | 1.31 | 2.24 | 2.49 |
| D [4, 3] | μm | 255.74 | 267.88 | 245.93 | 286.44 | 203.07 | 80.01 | 86.72 |
| Type of | | Unimodal | Unimodal | Unimodal | Unimodal | Unimodal | Unimodal | Unimodal |
| distribution | | | | | | | | |
| DCM | ppm | 60819 | 59223 | 63204 | 64934 | 68804 | 50612 | 39906 |
| Acetone | ppm | 60 | 63 | 77 | 68 | 71 | 102 | 111 |
| Ethyl acetate | ppm | 5 | 5 | 5 | 5 | 6 | 350 | 395 |

^{a)}F_solids (=_feed × C_feed) is the flow of solid material fed to the spray dryer.
^{b)}Yields have a large error, as the dryer was not cleaned between tests.

### Example 6

This example provides the results of experiments in which a dispersion of VX-950 prepared by fluidized spray drying was directly compressed into a tablet.

### Introduction

Tableting properties can be affected by many factors such as physical-chemical and mechanical properties of API, related excipients, and process parameters. To achieve robust formulation, these effects are evaluated during the formulation development stage. These experiments evaluated the effects of a dispersion spray dried via fluidized spray drying with different methods of Vitamin E addition (spray congealed, BASF Vit E acetate, melt granulated onto excipients, and melt granulated onto the dispersion). Tableting properties were characterized by tablet hardness, ejection force, and thickness.

### Methods

The addition of different types of Vit E and different processes for the addition of the Vit E were evaluated. The types of Vit E and methods of addition to the dispersion are shown below.

A dispersion of VX-950 was prepared by fluidized spray drying as described herein.

TABLE 13
VX950 SD Tableting Experiment Design (Potency: 250 mg VX950)

| Trial # | Vit E type | Vit E type |
|---|---|---|
| A | VitE-TPGS (24 mg) | Granulated VitE on excipients |
| C | VitE-Acetate (48 mg) | Used as is |
| E | Vit E-TPGS(24 mg) | Vit E Spray Congealed |
| F | Vit E-TPGS (24 mg) | Granulated Vit E onto VX950 |

TABLE 14
Trial# A Formulation

| | | Wt/Tablet | | Theoretical |
|---|---|---|---|---|
| Item | Ingredients | (mg) | % | Qt. (g) |
| | Physical mixture | | | |
| 1 | Solid Dispersion | 339.9 | 66.32 | 19.90 |
| | (73.55% VX950/26.45% | | | |
| | HPMCAS) | | | |
| 2 | PHARMATOSE ® DCL 22 | 37.5 | 7.32 | 2.20 |
| | (Lactose) | | | |
| 3 | AC-DI-SOL ® (Cross | 24.0 | 4.68 | 1.40 |
| | carmellose sodium) | | | |
| 4 | Sodium Stearyl Fumarate | 1.6 | 0.32 | 0.10 |
| 5 | SLS | 3.4 | 0.66 | 0.20 |
| 6 | AVICEL ® pH 113 | 33.7 | 6.58 | 1.97 |
| | (Microcrystalline cellulo [symbol] | | | |
| 7 | Vitamin E TPGS | 24.0 | 4.68 | 1.40 |
| | (granulated on excipients) | | | |
| 8 | AC-DI-SOL ® (Cross | 16.0 | 3.12 | 0.94 |
| | carmellose sodium) | | | |
| 9 | Cabosil M-5 (Colloidal | 8.0 | 1.56 | 0.47 |
| | silicon dioxide) | | | |
| 10 | Sodium Stearyl Fumarate | 24.4 | 4.76 | 1.43 |
| | Total | 512.5 | 100 | 30.00 |

Note:
VX 950 SD Lot 02
Potency: 250 mg VX950 [symbol]
indicates data missing or illegible when filed

TABLE 15
Trial# C Formulation

| | | Wt/Tablet | | Theoretical |
|---|---|---|---|---|
| Item | Ingredients | (mg) | % | Qt. (g) |
| | Physical mixture | | | |
| 1 | Solid Dispersion | 339.9 | 63.36 | 79.19 |
| | (73.55% VX950/26.45% | | | |
| | HPMCAS) | | | |
| 2 | PHARMATOSE ® DCL 22 | 37.5 | 6.99 | 8.74 |
| | (Lactose) | | | |
| 3 | AC-DI-SOL ® (Cross | 24.0 | 4.47 | 5.59 |
| | carmellose sodium) | | | |
| 4 | Sodium Stearyl Fumarate | 1.6 | 0.30 | 0.38 |
| 5 | SLS | 3.4 | 0.63 | 0.79 |
| 6 | AVICEL ® pH 113 | 33.7 | 6.28 | 7.85 |
| | (Microcrystalline cellulose) | | | |
| 7 | Vitamin E-Acetate | 48.0 | 8.95 | 11.18 |
| 8 | AC-DI-SOL ® (Cross | 16.0 | 2.98 | 3.73 |
| | carmellose sodium) | | | |
| 9 | Cabosil M-5 (Colloidal | 8.0 | 1.49 | 1.86 |
| | silicon dioxide) | | | |
| 10 | Sodium Stearyl Fumarate | 24.4 | 4.54 | 5.68 |
| | Total | 536.5 | 100 | 125.00 |

TABLE 16
Trial# E Formulation

| | | Wt/Tablet | | Theoretical |
|---|---|---|---|---|
| Item | Ingredients | (mg) | % | Qt. (g) |
| | Physical mixture | | | |
| 1 | Solid Dispersion | 339.9 | 66.32 | 82.90 |
| | (73.55% VX950/26.45% | | | |
| | HPMCAS) | | | |
| 2 | PHARMATOSE ® DCL 22 | 37.5 | 7.32 | 9.15 |
| | (Lactose) | | | |
| 3 | AC-DI-SOL ® (Cross | 24.0 | 4.68 | 5.85 |
| | carmellose sodium) | | | |
| 4 | Sodium Stearyl Fumarate | 1.6 | 0.32 | 0.40 |
| 5 | SLS | 3.4 | 0.66 | 0.83 |
| 6 | AVICEL ® pH 113 | 33.7 | 6.58 | 8.22 |
| | (Microcrystalline cellulose) | | | |
| 7 | Vitamin E Spray Congealed | 24.0 | 4.68 | 5.85 |
| 8 | AC-DI-SOL ® (Cross | 16.0 | 3.12 | 3.90 |
| | carmellose sodium) | | | |
| 9 | Cabosil M-5 (Colloidal | 8.0 | 1.56 | 1.95 |
| | silicon dioxide) | | | |
| 10 | Sodium Stearyl Fumarate | 24.4 | 4.76 | 5.95 |
| | Total | 512.5 | 100 | 125.00 |

Note:
VX 950 SD Lot 02
Potency: 250 mg VX950

TABLE 17
Trial# F Formulation

| | | Wt/Tablet | | Theoretical |
|---|---|---|---|---|
| Item | Ingredients | (mg) | % | Qt. (g) |
| 1 | Solid Dispersion | 339.9 | 66.32 | 66.32 |
| | (73.55% VX950/26.45% | | | |
| | HPMCAS) | | | |
| 2 | Vitamin E granulated onto | 24.0 | 4.68 | 4.68 |
| | dispersion | | | |
| 3 | PHARMATOSE ® DCL 22 | 37.5 | 7.32 | 7.32 |
| | (Lactose) | | | |
| 4 | AC-DI-SOL ® (Cross | 24.0 | 4.68 | 4.68 |
| | carmellose sodium) | | | |
| 5 | Sodium Stearyl Fumarate | 1.6 | 0.32 | 0.32 |
| 6 | SLS | 3.4 | 0.66 | 0.66 |
| 7 | AVICEL ® pH 113 | 33.7 | 6.58 | 6.58 |
| | (Microcrystalline cellulose) | | | |
| 8 | AC-DI-SOL ® (Cross | 16.0 | 3.12 | 3.12 |
| | carmellose sodium) | | | |
| 9 | Cabosil M-5 (Colloidal | 8.0 | 1.56 | 1.56 |
| | silicon dioxide) | | | |
| 10 | Sodium Stearyl Fumarate | 24.4 | 4.76 | 4.76 |
| | Total | 512.5 | 100 | 100.00 |

Note:
VX 950 SD Lot 02
Potency: 250 mg VX950

TABLE 18
VX 950 SD Lot 02 Physical parameters

| | |
|---|---|
| D10 (μm) | 13.37 |
| D50 (μm) | 51.45 |
| D90 (μm) | 141.67 |
| Bulk density (g/ml) | 0.25 |
| Tap density (g/ml) | 0.32 |

### Results

TABLE 19
Results from the Compression Run

| | | | | Tooling shape: | | | | | |
|---|---|---|---|---|---|---|---|---|---|
| | | | | oval 0.6250 in.* | | | | | |
| #A-GranExcp-DC | Tablet wt. = 512.5 mg | | | 0.3750 in. | | | | | |
| | Run #1 | | Run #2 | Run #3 | Run #4 | Run #5 | Run #6 | Run #7 | Run #8 |
| Compress force | 2.37 | | 2.7 | 2.84 | 4.43 | 6.95 | 8.4 | 12.5 | 16.01 |
| (kN) | | | | | | | | | |
| Eject (N) | 70 | | 83 | 83 | 86 | 90 | 90 | 90 | 90 |
| Hardness (kp) | 3 | | 3.2 | 3.3 | 4.8 | 6.5 | 8.5 | 10.5 | 10.8 |
| Thickness (mm) | 6.97 | | 6.46 | 6.43 | 6.02 | 5.76 | 5.51 | 5.35 | 5.30 |
| | | | | Tooling shape: | | | | | |
| | | | | oval 0.6250 in.* | | | | | |
| #C-Acet-DC | Tablet wt. = 536.5 mg | | | 0.3750 in. | | | | | |
| | Run #1 | Run #2 | Run #3 | Run #4 | Run #5 | Run #6 | Run #7 | Run #8 | Run #9 |
| Compress force | 1.1 | 1.9 | 2.3 | 2.8 | 4.4 | 6.8 | 10.1 | 13.4 | 18.1 |
| (kN) | | | | | | | | | |
| Eject (N) | 83 | 83 | | | | | | | |
| Hardness (kp) | N/A | 2.3 | 2.5 | 2.7 | 4.4 | 6.5 | 9.9 | 13.3 | 14.5 |
| Thickness (mm) | 8.30 | 7.70 | 7.38 | 7.05 | 6.48 | 6.03 | 5.76 | 5.60 | 5.52 |

| | | | Tooling shape: | | | | |
|---|---|---|---|---|---|---|---|
| | | | oval 0.6250 in.* | | | | |
| #E-SpCong-DC | Tablet wt. = 512.5 mg | | 0.3750 in. | | | | |
| | Run #1 | Run #2 | Run #3 | Run #4 | Run #5 | Run #6 | Run #7 |
| Compress force | 1.77 | 2.23 | 3.68 | 5.61 | 8.8 | 15.5 | 18.09 |
| (kN) | | | | | | | |
| Eject (N) | 83 | 83 | 90 | 95 | 120 | 95 | 95.00 |
| Hardness (kp) | 2 | 2.4 | 3.6 | 6.1 | 10.4 | 14 | 14.23 |
| Thickness (mm) | 7.46 | 7.06 | 6.42 | 6.01 | 5.53 | 5.33 | 5.29 |
| | | | Tooling shape: | | | | |
| #F-Gran | | | oval 0.6250 in.* | | | | |
| VX950-DC | Tablet wt. = 512.5 mg | | 0.3750 in. | | | | |
| | Run #1 | Run #2 | Run #3 | Run #4 | Run #5 | Run #6 | Run #7 |
| Compress force | 2.18 | 3.37 | 4.41 | 6.27 | 10.28 | 12.8 | 18.83 |
| (kN) | | | | | | | |
| Eject (N) | 75 | 83 | 83 | 85 | 90 | 90 | 90 |
| Hardness (kp) | 1.5 | 3.4 | 5.6 | 7.8 | 11.2 | 13.8 | 15.6 |
| Thickness (mm) | 6.98 | 6.44 | 6.00 | 5.81 | 5.55 | 5.37 | 5.28 |

TABLE 20
Blend Properties

| Flowability test | Flow index | Carr index |
|---|---|---|
| #A-GranExcp-DC | 9 | 31.1 |
| #C-Acet-DC | 14 | 34.9 |
| #E-SpCong-DC | 12 | 29.3 |
| #F-GranVX950-DC | 12 | 41.0 |
| Bulk/Tap Density | Bulk (g/ml) | Tap (g/ml) |
| #A-GranExcp-DC | 0.31 | 0.46 |
| #C-Acet-DC | 0.28 | 0.43 |
| #E-SpCong-DC | 0.31 | 0.43 |
| #F-GranVX950-DC | 0.36 | 0.61 |

### Example 7

A solid dispersion was prepared comprising the following ingredients (percentage of total weight):

| | |
|---|---|
| VX-950 | 49.5% |
| HPMC 40 cp | 49.5% |
| SLS | 1% |

The composition 1 was prepared by dissolving VX-950, HPMC, and SLS in methanol:methylene chloride (1:1) followed by evaporation of the solvents using rotation evaporation under vacuum. The product was milled to particles with mean particle size of about 200 μm.

### Example 8

A solid dispersion was prepared comprising the following ingredients (percentage of total weight):

| | |
|---|---|
| VX-950 | 49.5% |
| HPC | 49.5% |
| SLS | 1% |

The composition 2 was prepared by dissolving VX-950 and HPC in methylene chloride. SLS was suspended in the solution. The solvent was then evaporated by rotation evaporation under vacuum. The product was milled to particles with mean particle size of about 200 μm.

### Example 9

A solid dispersion was prepared comprising the following ingredients (percentage of total weight):

| | |
|---|---|
| VX-950 | 49.5% |
| PVP K30 | 49.5% |
| SLS | 1% |

The composition 3 was prepared by dissolving VX-950, PVP K30, and suspending SLS in methanol:methylene chloride followed by spray-drying to remove the solvent. The mean particle size of the product is about 150 μm.

### Example 10

A solid dispersion was prepared comprising the following ingredients (percentage of total weight):

| | |
|---|---|
| VX-950 | 49.5% |
| HPMCP | 49.5% |
| SLS | 1% |

The composition 4 was prepared by using a similar procedure as in example 3. The mean particle size of the product is about 150 μm.

Other types of polymers and surfactants were also tested (see the following examples). The ratio of VX-950 and the polymers and the amount of surfactants were also tested in various assays (see the following examples).

### Example 11

Various compositions of VX-950 were tested in a rat pharmacokinetic (PK) assay.

TABLE 21
Rat Pharmacokinetic data
RAT PK

| | | |
|---|---|---|
| | Systemic | Portal |
| Dose oral | Plasma | Plasma |
| (mg/kg) | F (%) | Fa (%) |

VX-950 Formulation

| | | | |
|---|---|---|---|
| 3 mg/ml Solution in Propylene Glycol | 30 | 2.4% | 15.2% |
| Crystalline Aqueous Suspension | 30 | 1.1% | 4.7% |
| 1% CMC 500 nm Nanosuspension | 30 | 1.7% | 4.0% |
| (crystalline), 3 mg/ml | | | |
| Amorphous Aqueous Suspension, | 30 | 0.4% | 1.4% |
| 3 mg/ml (not a solid dispersion) | | | |

Solid Dispersions

| | | | |
|---|---|---|---|
| 10% VX-950/10% PEG300/10% SLS/ | 30 | 41.1% | 104.4% |
| PVP-K30 solvent = EtOH, | | | |
| aqueous dose | | | |
| 10% VX-950/5% SLS/42.5% PVP-K30/ | 30 | 19.6% | 77.6% |
| PEG8000, solvent = EtOH, | | | |
| aqueous dose | | | |
| 10% VX-950/10% NMP/10% SLS/ | 30 | 32.3% | 73.4% |
| PVP-K30, solvent = EtOH, | | | |
| aqueous dose | | | |
| 10% VX-950/10% PEG300/10% SLS/ | 30 | 12.7% | 26.6% |
| PVP-K30, solvent = MeCl/EtOH, | | | |
| aqueous dose | | | |
| 10% VX-950 solvent = molten PEG- | 30 | 5.6% | 24.3% |
| 8000, aqueous dose | | | |

### Example 12

Various compositions of VX-950 were tested in a dog pharmacokinetic assay. In this study, the VX-950 compound tested was a 60:40 (+/−5%) mixture of L:D isomers.

TABLE 22
Pharmacokinetic parameters of VX-950 D/L mixture
(in dog; 15 mg/kg dose)

| | | Cₘₐₓ | Tₘₐₓ | T_{1/2} | |
|---|---|---|---|---|---|
| Formulation | % F | μg/ml | hr | hr | |
| 20% VRT108720/77% PVP K30/ | 15.12 | 0.89 | 1.33 | 2.25 | Mean |
| 3% SLS | 53.85 | 66 | 43 | 31 | CV % |
| solid dispersion (EtOH) | | | | | |
| 25% VRT108720/72% PVPK30/ | 5.81 | 0.38 | 1.17 | 1.82 | Mean |
| 3% SLS | 20 | 37 | 25 | 34 | CV % |
| 33% VRT108720/64% PVPK30/ | 7.75 | 0.47 | 0.58 | 2.52 | Mean |
| 3% SLS | 69.28 | 63 | 65 | 22 | CV % |
| Spray-drying | | | | | |
| 50% VRT108720/47% PVPK30/ | 18.22 | 1.19 | 1.33 | 2.28 | Mean |
| 3% SLS | 38.47 | 41 | 43 | 16 | CV % |
| Spray-drying | | | | | |
| 20% VRT108720/5% Pluronic | 25.19 | 1.74 | 1.17 | 4.42 | Mean |
| F68/75% Kollidon VA64 | 39.79 | 61 | 49 | 22 | CV % |
| melt dispersion | | | | | |
| 20% VRT108720/5% Labrasol/ | 3.49 | 0.07 | 1.67 | 1.19 | Mean |
| 75% Kollidon VA64 | 47.14 | 42 | 35 | 3 | CV % |
| melt dispersion | | | | | |
| 20% VRT108720/5% Capryol/ | 13.57 | 0.82 | 1 | 1.12 | Mean |
| 75% Kollidon VA64 | 77.78 | 41 | 50 | 32 | CV % |
| melt dispersion | | | | | |
| 20% VRT108720/5% Cremophor/ | 8.91 | 0.63 | 0.75 | 2.34 | Mean |
| 75% Kollidon VA64 | 39.85 | 21 | 88 | 40 | CV % |
| melt dispersion | | | | | |
| 20% VRT108720/5% SLS/75% | 1.55 | 0.13 | 1 | 1.05 | Mean |
| Kollidon VA64 | 43.3 | 61 | 50 | 75 | CV % |
| melt dispersion | | | | | |

### Example 13

The physical stability of various compositions were tested. The results are in Table 23 below.

TABLE 23
Physical stability data
Physical Stability of VX-950 Solid Dispersions

| | | | A = amorphous | | | | |
|---|---|---|---|---|---|---|---|
| | | | C = crystalline | | | | |
| Formulation | | | Blank = not tested | | | | |
| Description | Condition | Lid | 0 | 1 wk | 2 wk | 1 mo | 2 mo |
| amorphous form of | 40 C/75% RH | Closed | A | A | | A | A |
| pure VX-950 (no | 60° C. | Closed | A | A | | A | A |
| polymer) | 25°° C./60% | Closed | A | A | | A | A |
| solvent evaporation, | RH | | | | | | |
| MeCl2 | 40° C./75% RH | Open | | | | C | |
| VX-950:PVP K30, | 40° C./75% RH | Closed | A | | | A | A |
| 1:1 | 60° C. | Closed | A | | | A | A |
| 1% SLS | | | | | | | |
| solvent evaporation, | | | | | | | |
| EtOH:MeCl2, 8:2 | | | | | | | |
| | 25°° C./60% | Closed | A | | | A | A |
| | RH | | | | | | |
| VX-950:PVP K30, | 40° C./75% RH | closed | A | | A | A | A |
| 1:1, | 60° C. | closed | A | | A | A | A |
| 1% SLS | 25° C./60% RH | closed | A | | A | A | A |
| spray-dried, | 40° C./75% RH | open | | | | C | |
| MeOH:acetone, 2:1 | | | | | | | |
| VX950:PVP K16, 1:1 | 40° C./75% RH | closed | A | A | | A | A |
| 1% SLS | 60° C. | closed | A | A | | A | A |
| Solvent evaporation, | 25° C./60% RH | closed | A | A | | A | A |
| MeCl2 | 40° C./75% RH | open | | | | A | |

### Example 14

The chiral stability of various compositions were tested. The results are in Table 24 below.

TABLE 24
Chiral stability data
Chiral Stability of
49.5% VX950, 1% SLS, 49.5% Polymer

| | Condition | | % AUC D- |
|---|---|---|---|
| Polymer | (sealed containers) | time | isomer |
| K16 | 25 C./60% RH | 5 mo | 22 |
| K16 | 40 C./75% RH | 5 mo | 28 |
| K30 | 25 C./60% RH | 5 mo | 3 |
| K30 | 40 C./75% RH | 5 mo | 7.5 |

### Example 15

The solubility of various compositions were tested. The results are in Table 25 below.

TABLE 25
Solubility data
Spray-dried Dispersions of VX-950
Absolute Solubility in Water (measured at 1 hr)

| | | | suspension | Absolute |
|---|---|---|---|---|
| | | Solid load | conc. | solubility, |
| Composition | Solvent | (g/ml) | mg/ml | μg/ml |
| VX950:PVPK30, 1:1, | MeCl2 | 40% | 50 | 66.87 |
| 2% Pluronic F108 | | | | |
| VX950:HPMC, 1:1, | MeCl2/t- | 10% | 50 | 399.7 |
| 2% SLS | BT, 1:1 | | | |
| VX950:PVPK30, 1:1, | MeOH/ | 10% | 10 | 41.22 |
| 2% Pluronic F108 | acetone, 2:1 | | | |
| VX-950:PVPK30, 1:1, | MeCl2 | 10% | 10 | 22.43 |
| 2% Pluronic F108 | | | | |
| VX-950:PVPK30, 1:1, | MeCl2 | 10% | 10 | 344.2 |
| 2% SLS | | | | |
| VX-950:PVPK16, 1:1, | MeCl2 | 10% | 10 | 277.2 |
| 2% SLS | | | | |
| VX-950:PVPK16, 1:2, | MeCl2 | 10% | 10 | 346.5 |
| 2% SLS | | | | |
| VX-950:PVPK16, 1:1, | MeCl2 | 10% | 10 | 367 |
| 1% SLS | | | | |
| VX-950:PVPK30, 1:1, | MeCl2 | 10% | 10 | 349.5 |
| 2% SLS | | | | |

### Example 16

The effect of SLS concentration on the apparent solubility of VX-950 solid dispersions were tested. The results are in Table 26 below.

TABLE 26
Solubility data
Effect of SLS concentration on the apparent
solubility of VX-950 solid dispersions

| VX-950 | % dissolved in |
|---|---|
| (95% L/5% D) | water @ 5 min. |
| No Excipients | 2.7 |
| Only PVP-K30 | 5.6 |
| 0.5% SLS 89.5% PVP | 32.6 |
| 1% SLS 89% PVP | 46.7 |
| 2% SLS 88% PVP | 37.7 |
| 3% SLS 87% PVP | 32.2 |

### Example 17

An oral dosage formulation was prepared as follows. VX-950 and PVP K29/32 were dissolved in methylene chloride, then sodium lauryl sulfate was added and dispersed in the solution to form a homogenous suspension. This suspension was spray-dried using an inlet temperature of 90° C. and an outlet temperature of 56° C., and the product was collected from the cyclone. The spray-dried'dispersion was fluid-bed, dried at 75° C. for 8 hours.

TABLE 27
VX-950 Solid Dispersion

| % (w/w) | Ingredient | |
|---|---|---|
| 49.5 | VX-950 | Spray-dried |
| 49.5 | PVP K29/32 | from a MeCl2 |
| 1 | SLS | solution |

The solid dispersion was suspended in a 1% HPMC, 0.002% simethicone solution using a steel rotary mixer. The resultant suspension is physically and chemically stable at the concentrations of 0.8-50 mg/ml VX-950 for at least 24 hours. The powder is then suspended and dosed within 24 hrs as described in the table below.

TABLE 28
Suspension Vehicle

| % | Ingredient | Function |
|---|---|---|
| 1 | Low viscosity hydroxypropyl | Suspending agent |
| | methylcellulose | |
| 0.002 | Simethicone | Anti-foam |
| 99 | Water | diluent |

### Example 18

Dispersions in single dose glass vials mixed with 1% HPMC vehicle were dosed. The solid residue remaining in the vial was 0.8%-4% compared to 28%-56% when dosed in a syringe mixed with water (January 20 dosing below). Dispersions dosed were: VX950/PVPK-30/SLS (tox. lot, refreshed), VX950/HPMCAS/SLS/SDBS (spray dried at ISP starting with crystalline DS containing 5% PVPK-30), VX950/HPMC E15/10% Vit E TPGS, VX950/PVP-VA110% Vit E TPGS. The results of these studies are provided below.

TABLE 29

| | Mean Cmax | Mean Tmax | |
|---|---|---|---|
| Formulation ID (30 mg/Kg dose) | (ng/mL) | (hr) | Mean % F |
| 1:1 VX950:PVPK30, 1% SLS | 981 ± 200 | 0.6 ± 0.3 | 19.6 ± 3.1 |
| (Refreshed Tox.) | | | |
| Niro-49% HPMCAS/1% SLS/ | 980 ± 200 | 0.9 ± 0.3 | 29.5 ± 4.8 |
| 1% SDBS/49% VX-950 | | | |
| 40.5% PVP-VA/10% ETPGS/ | 1482 ± 400 | 0.5 ± 0.0 | 29.8 ± 9.1 |
| 49.5% VX-950 | | | |
| 40.5% HPMC/10% ETPGS/ | 1890 ± 400 | 0.4 ± 0.1 | 34.7 ± 7.8 |
| 49.5% VX-950 | | | |

As can be seen in the above table, HPMC E-15/10% Vit ETPGS had the highest Cmax and % F. PVP-VA/10% Vit ETPGS had the second highest Cmax and % F. HPMCAS exhibited a somewhat sustained release profile with a Cmax comparable to PVPK-30 refreshed dispersion and a % F comparable to PVP-VA.

### Example 19

Three formulations were manufactured on the SD Micro spray drier (100 gm). The first 2 formulations had the same ingredients, but varied in acetone levels. The third formulation was a polymer mixture of HPC and HPMC phthalate (2:1). All three formulations contained 1% SLS and 1% SDBS and drug substance that had 5% PVPK-30.

Dissolution of the polymers required homogenization, and all 3 formulations spray-dried very easily. All formulations had detectible residual solvents after manufacture, but both solvents were easily removed with oven drying (60° C.). The addition of acetone appeared to have lowered the initial content of methylene chloride. Residual solvents levels are summarized below

TABLE 30
Residual solvents from dispersions manufacture at ISP (100 gm scale)

| | | | | Residual | | | |
|---|---|---|---|---|---|---|---|
| | | | | Methylene | | Residual | |
| | | | Drying Time | Chloride | | Acetone | |
| Lot # | Formulation | solvent Ratio | (hr) | (ppm) | | (ppm) | |
| 2702-801 | 49% VX950, 49% | 100% | 0 | 10064 | | <100 | ppm |
| | HPMCAS, 1% | methylene | 1 | 114 | | <100 | ppm |
| | SLS, 1% SDBS | Chloride | 2 | <100 | ppm | <100 | ppm |
| | | | 63 | <100 | ppm | <100 | ppm |
| 2702-802 | 49% VX950, 49% | 30% Acetone/ | 0 | 2889 | | 1869 | |
| | HPMCAS, 1% | 70% | 1 | <100 | ppm | <100 | ppm |
| | SLS, 1% SDBS | methylene | 2 | <100 | ppm | <100 | ppm |
| | | chloride | 63 | <100 | ppm | <100 | ppm |
| 2702-803 | 49% VX950, 16% | 30% Acetone/ | 0 | 5641 | | <100 | ppm |
| | HPPh, 33% HPC, | 70% | 1 | <100 | ppm | <100 | ppm |
| | 1% SLS, 1% | methylene | 2 | <100 | ppm | <100 | ppm |
| | SDBS | chloride | 63 | <100 | ppm | <100 | ppm |

### Example 20

A liquid dispersion including HPMCE 50/1% SLS was explored extensively as a suspension in several vehicles at room temperature or refrigerated conditions as follows:

1. 1% HPMC vehicle with varying levels of Vit E TPGS at VX950 concentration of 3 mg/mL.

Solubility and physical stability of the HPMC E50/1% SLS dispersion in suspension containing 0.067%, 1%, 5%, and 10% Vit E TPGS were evaluated using HPLC and XRD according to several procedures to simulate the dosings in the actual tox. studies (b.i.d. dosing, 8-12 hours apart).

Procedure 1: Suspensions made and stored at RT and evaluated at 1, 3, 24, 48 hrs (stirring for 3 hours then stored unstirred until the 24 hrs time point where they're stirred for 15 minutes before sampling).

Procedure 2: Suspensions made at RT but stored at 5° C. after 3 hrs unstirred. At the 24 time point, suspensions were stirred at 5° C. (in ice) before sampling.

Procedure 3: Suspensions made at RT but stored at 5° C. after 3 hrs unstirred. At the 24 time point, suspensions were stirred for 15 minutes at RT (warmed-up) before sampling.

Procedure 4: evaluated only for the 10% Vit E TPGS containing vehicle. Suspensions made and stored at 5° C. and evaluated at 1, 3, 24, 48 hrs (stirring for 3 hours then stored unstirred until the 24 hrs time point where they're stirred for 15 minutes in ice before sampling)

For all the above, kinetic solubility in simulated intestinal fluid at 37° C. was evaluated 1 hr after preparation and after 24 hours of storage under the conditions above.

Results:

A. Procedure 1: Solubility increases as a function of % Vit E TPGS (at 1 and 3 hrs). A significant decrease in solubility is observed after 1 hr for suspensions with the higher levels of Vit E TPGS (10% and 5%) although the actual solubility values remained high 600-700 Tg/mL. Collected solid residues dried for 24-48 hrs exhibited some crystallinity. A slight decrease in solubility was observed for the suspension containing 1% Vit E TPGS as well as slight crystallinity. No decrease was observed at the 0.067% Vit E TPGS level and solid residue was amorphous.
Procedure 2: No decrease (change) in solubility was observed at any of the Vit E TPGS levels.
Procedure 3 (warming up): No decrease (change) in solubility was observed at any of the Vit E TPGS levels and the values were the same as in procedure 2
Procedure 4: At 1 and 3 hrs, solubility was lower as compared to procedure 2 (i.e. when made at 5° C. vs at RT), probably due to retarded diffusion/higher viscosity at the lower temperature. No decrease in solubility was observed over 48 hrs and the values were comparable to those obtained in procedure 2 after 24 hrs.

B. Procedure 1, after 1 hr: A significant decrease in solubility is observed at the 10% Vit E TPGS level after 1 hr and a slight decrease is observed at the 5% Vit E TPGS level only after 3 hrs. No decrease was observed at the lower levels (1% and 0.067%) over 5 hrs. In comparison, the suspension containing 10% Vit E TPGS made and stirred on ice (5° C.) for 1 hr shows no decrease in solubility over 5 hrs, however, the actual solubility value is significantly lower than that made at RT. This may explain the reduced % F for the latter in rats.
Procedure 1, after 24 hrs: In comparison to the suspension made and evaluated after 1 hr, the solubility/dissolution is significantly lower for the 1% and 5% Vit E TPGS levels. The 0.067% suspension exhibited initial solubility similar to that observed for the freshly prepared suspension (tested after 1 hr), however a slight decrease in solubility was observed after 2 hrs in SIF, which was not observed for the fresh suspension.
Procedure 2, 24 hrs: similar results as observed for procedure 1 where the suspensions containing lower % Vit E TPGS (0.067% and 1%) showed no decrease in solubility/dissolution after 5 hrs and the absolute values were also the same as those when tested 1 hr after preparation

Conclusions: from the suspension solubility and the kinetic solubility in SIF at 37° C., the suspension containing 0.067% Vit E TPGS exhibited no change in performance (no decrease in suspension solubility over 24 hrs and no decrease in dissolution over 5 hrs for a fresh and a 24 hrs old sample) whether stored at RT or at 5° C. Similar behavior was observed for the suspensions containing 1% and 5% Vit E TPGS only if stored at 5° C. (made at RT).

A gradual decrease in kinetic solubility in SIF at 37° C. was observed over 5 hours for 24 hrs old samples after storage at 5° C. whether warmed to RT or not before evaluation. The suspension made at 5° C. showed lower dissolution/solubility in SIF when evaluated 1 hr after preparation compared to 24 hrs probably due to continued dissolution during storage at 5° C.

### Example 21

A mixture of the following components was spray dried to provide a solid dispersion of VX-950. VX-950/HPMCAS-HG/SLS was combined in a ratio of 49.5/49.5/1 wt/wt and combined in a solvent system at a solid concentration of 10, where the solvent system included methylene chloride/acetone/glacial acetic acid in a ratio of 66.6/28.5/5 to provide a product having a d50 of 43.03 and a bulk density of 0.37.

### Example 22

A mixture of the following components was spray dried to provide a solid dispersion of VX-950. VX-950/HPMCAS-HG/SLS was combined in a ratio of 49.5/49.5/1 wt/wt and combined in a solvent system at a solid concentration of 10, where the solvent system included methylene chloride/acetone/glacial acetic acid in a ratio of 63/27/10 to provide a product having a d50 of 47.02 and a bulk density of 0.41.

### Example 23

Spray dried dispersions of VX-950 were prepared using with multiple VX-950 lots, HPMCAS-HG (Hypromellose Acetate Succinate, HG grade, Shin-Etsu Chemical Co.) polymer, and SLS (Sodium Lauryl Sulfate, Fisher) surfactant. Spray drying and subsequent post-drying in a biconical dryer were performed. Dry dispersion with low residual solvent levels and target powder properties were manufactured. Success criteria included having acceptable process yield (>80%), and meeting all target drug product specifications for purity, and matching the target properties within the range specified for physical characteristics (particle size and bulk density).

### Formulation Composition and Process Outline

The overall formulation composition for each of two active dispersion manufactures is described in Table 31.

TABLE 31
Formulation composition of each of the two active dispersion
manufactures based off of 116.25 kg VX-950 at 13 wt %.

| Component Function | Component | kg |
|---|---|---|
| API | VX-950 | 116.25 |
| Polymer/Dispersant | Hypromellose Acetate Succinate, | 116.25 |
| | NF/JPE (HPMCAS-HG) | |
| Surfactant | Sodium Lauryl Sulfate, NF (SLS) | 2.348 |
| Process Solvent | Methylene Chloride, NF | 1178.8 |
| | (for Dispersion) | |
| Process Solvent | Acetone, NF | 377.2 |
| | (for Dispersion) | |
| Process Solvent | DI Water | 15.7 |

An explanation of the process flow is below:

A) Preparation of Solution and Spray Dryer

1) Methylene chloride was prepared in the equilibration solvent tank.

2) 100 kg of the prescribed acetone amount was added to the mixing reactor (refer to Table 31).

3) Methylene chloride at the appropriate amount (refer to Table 31) was prepared in the main solution reactor. Differential pressure cells confirmed the correct amounts of charged solvents.

4) VX-950 drug substance was charged into the main solution reactor (refer to Table 31). The overall solids loading was at 13 wt %. A sample was taken to verify the drug substance was dissolved by visual inspection.

5) HPMCAS-HG was charged into the main solution reactor (refer to Table 31). The overall solids loading were at 13 wt %.

6) The remaining prescribed acetone amount was added to the mixing reactor (refer to Table 31).

7) The acetone, SLS, and DI water were charged into the main solution reactor.

8) The resultant batch was tested for visual appearance and viscosity once dissolved.

9) The Spraying Systems SK-MFP pressure nozzle was installed and tested for correct atomization with the equilibration solvent. (Nozzles 48/21, 50/21, or 52/21 can also be used.)

B) Start-up of the Spray Dryer

1) The spray dryer was heated to the appropriate outlet temperature.

2) Equilibration solvents were sprayed until all parameters are equilibrated and constant.

3) Spray drying of the feed solution was commenced once the spray dryer was equilibrated.

4) Dry particles were inertially separated from the process gas by a cyclone and collected within polyethylene bags. The process gas was then filtered for fine particles and condensed to remove process solvents.

5) Initial sample was taken and tested for particle size distribution and bulk and tap densities.
a) If particle size distribution and densities were within acceptance criteria and near targets, the process continued and samples were taken per the sampling plan.
b) If particle size distribution and densities were not within acceptance criteria and not near targets, the process was optimized (e.g., by changing one or more of the following: nozzle, outlet temperature, feed pressure) as needed. Collection bags were changed and the powder outside of the acceptance criteria was held in quarantine. Once the sample was within specification, the process with current parameters was started.

C) On-going Spray Drying

1) Took samples per sampling plan.

2) Noted any changes to the processing parameters.

3) Noted any stoppages or out of continuous operation occurrences.

4) Upon completion of spray drying the feed solution, switched to equilibration solvent and followed normal shut down procedures.

D) Post-Drying Process

1) Spray dried dispersion was charged into a secondary dryer and dried until all residual solvents (methylene chloride, acetone, ethyl acetate, and toluene) were below the specifications established.

### Equipment

An 8000-L industrial scale reactor equipped with a mechanical stirrer and thermal circuit was used for mixing of the initial solution. An industrial scale spray dryer (Niro Pharmaceutical Spray Dryer FSD12.5CC) was used in normal co-current spray drying mode. A pressure nozzle system (Spraying Systems Maximum Free Passage SK-MFP Series variety, orifice 48-54, core 21) was utilized. A high performance pressure pump with solvent-compatible/resistant gaskets pumped the feed solution through the atomizer into the spray drying vessel. An inertial cyclone separated the product from the process gas and solvent vapors. A filter bag then collected the fine particles not separated by the cyclone. The resultant gas was condensed to remove process solvents and recycled back to the heater and spray dryer (closed cycle).

The resultant product was transferred to a biconical vacuum dryer for drying of residual solvents.

### Key Process Controls and Parameters

Key process controls and parameters were needed for both the spray drying and biconical drying process. The primary process controls parameters have been identified through preliminary research batches.

Key process controls and parameters for the spray drying process, which were monitored and recorded over the entirety of the run time, were:
Atomizer/nozzle Installed
Feed Pressure
Inlet Temperature
Condenser Temperature Set Point (at about −10 to −15° C.)

Key process metrics for the spray drying process, which were monitored and recorded over the entirety of the run time, were:
Solution Feed Rate
Outlet Temperature
Cyclone Pressure Differential and Drying Gas Flow Rate

Table 32 defines spray drying process parameters/metrics, settings/ranges, and target guidelines.

TABLE 32
Spray drying variables, settings, and targets

| Variable | Setting/Range |
|---|---|
| Atomizer Installed | Spray Systems SK-MFP |
| Solution Feedrate | 120-200 kg/hr |
| Feed Pressure | 20-50 bar |
| Inlet Temperature | 50-80° C. |
| Outlet Temperature | 25-31° C. |
| Cyclone Pressure | 10.5-13.5 cm H₂0 |
| Differential | |

### Materials

An excipients and process solvents used complied with the current monographs of the European Pharmacopoeia, the Japanese Pharmacopoeia or the USP/NF, as indicated in Tables 31 and 33. All excipients and process solvents were purchased from approved suppliers. Manufacturer certificates of analysis were accepted and all materials received will undergo testing.

TABLE 33
Materials

| Material | Source |
|---|---|
| VX-950 | |
| Hypromellose Acetate | Shin-Etsu Chemical Co. |
| Succinate, NF/JPE | |
| (HPMCAS) (Aqoat AS- | |
| HG) | |
| Sodium Lauryl Sulfate | Sigma/Fisher |
| (SLS), NF | |
| Methylene Chloride, NF | |
| Acetone, NF | |
| DI Water | |

### Variations in Manufacture

Manufacture 2 used a process optimized for dispersion. Most notably this dispersion had larger particle size and bulk density than Manufacture 1, as needed for enhanced powder flowability and direct compression on a high-speed tablet press. Spray drying parameters were varied to make such powder. Variations were also made to tighten the process and to avoid possible deviations.

### Example 24

Spray dried dispersions of VX-950 were prepared using a solvent system that contained water, as described. The solvent system contained 75% methylene chloride; 24% acetone; and 1% water (w/w/w). The dispersions contained 49.5% VX-950; 49.5% HPMCAS-HG; and 1% SLS (w/w/w). Various combinations of outlet temperature, feed pressure, cyclone pressure, condenser setpoint temperature, nozzle type, solids loading, and solution feedrate were tested in the spray drying process. Varying these parameters varied the properties (particle size (PS)), span, bulk density, tap density, and levels of residual solvents) of the resulting dispersions.

### Example 25

### Objectives and Success Criteria

Dry dispersion with low residual solvent levels and target powder properties are manufactured. Success criteria include having acceptable process yield (>80%), and meeting all target drug product specifications for purity, and matching the target properties within the range specified for physical characteristics (particle size and bulk density).

### Formulation Composition and Process Outline

The overall formulation composition for the two active dispersion manufactures is described in Table 34.

TABLE 34
Formulation composition of the first active dispersion
manufacture based off of 100 kg VX-950 at 15 wt %.

| Component Function | Component | kg |
|---|---|---|
| API | VX-950 | 200.0 |
| Polymer/Dispersant | Hypromellose Acetate | 100.0 |
| | Succinate, NF/JPE | |
| | (HPMCAS-HG) | |
| Surfactant | Sodium Lauryl Sulfate, | 2.02 |
| | NF (SLS) | |
| Process Solvent | Methylene Chloride, NF | 858.6 |
| | (for Dispersion) | |
| Process Solvent | Acetone, NF | 274.7 |
| | (for Dispersion) | |
| Process Solvent | DI Water | 11.4 |

An explanation of the process flow is below:

A) Preparation of Solution and Spray Dryer
1) Methylene chloride is prepared in the equilibration solvent tank.
2) DI water is charged into a secondary mixing vessel (refer to Table 34).
3) Methylene chloride at the appropriate amount (refer to Table 34) is prepared in the main solution reactor. Differential pressure cells confirm the correct amounts of charged solvents.
4) VX-950 drug substance is charged into the main solution reactor. The overall solids loading are at 15 wt %. A sample is taken to verify the drug substance is dissolved by visual inspection.
5) HPMCAS-HG is charged into the main solution reactor (refer to Table 34). The overall solids loading is at 15 wt %.
6) The acetone amount is added to the mixing reactor (refer to Table 34). A sample is taken to determine if all solids are dissolved.
7) The SLS and water are added to the main mixing reactor.
8) The Spraying Systems SK-MFP pressure nozzle is installed and tested for correct atomization with the equilibration solvent.

B) Start-up of the Spray Dryer
1) The spray dryer is heated to the appropriate outlet temperature.
2) Equilibration solvents are sprayed until all parameters are equilibrated and constant.
3) Spray drying of the feed solution is commenced once the spray dryer is equilibrated.
4) Dry particles are inertially separated from the process gas by a cyclone and collected within polyethylene bags. The process gas is then filtered for fine particles and condensed to remove process solvents.
5) Initial sample is taken and tested for particle size distribution and bulk and tap densities.
a) If particle size distribution and densities are within acceptance criteria and near targets, the process continues and samples are taken per the sampling plan.
b) If particle size distribution and densities are not within acceptance criteria and not near targets, the process is optimized (by changing one or more of the following: outlet temperature, feed pressure, or condenser temperature as needed. Collection bags are changed and the powder outside of the acceptance criteria is held in quarantine. Once the sample is within specification, start the process with current parameters.

C) Post-Drying Process
1) Spray dried dispersion is charged into a secondary dryer.
2) This continues until all residual solvents (methylene chloride, acetone, ethyl acetate, and toluene) are below the specifications established.

D) Testing, Shipment
1) Samples of this dispersion are tested for release testing.

### Equipment

An 8000-L industrial scale reactor (R240) equipped with a mechanical stirrer and thermal circuit is used for mixing of the initial solution. A reactor (R32) is used for the SLS and water mixture. An industrial scale spray dryer (Niro Pharmaceutical Spray Dryer FSDI2.5CC) is used in normal co-current spray drying mode. A pressure nozzle system (Spraying Systems Maximum Free Passage SK-MFP Series variety, orifice 54, core 21) is utilized. A high performance pressure pump with solvent-compatible/resistant gaskets pumps the feed solution through the atomizer into the spray drying vessel. An inertial cyclone separates the product from the process gas and solvent vapors. A filter bag then collects the fine particles not separated by the cyclone. The resultant gas is condensed to remove process solvents and recycled back to the heater and spray dryer (closed cycle).

The resultant product is transferred to a biconical vacuum dryer (S901) for drying of residual solvents. The dry product is sieved within a nitrogen swept glovebox and packaged.

### Key Process Controls and Parameters

Key process controls and parameters are needed for both the spray drying and biconical drying process. The primary process controls parameters have been identified through preliminary research batches.

Key process controls and parameters for the spray drying process, which need to be monitored and recorded over the entirety of the run time, are:
Atomizer/nozzle Installed

Feed Pressure

Inlet Temperature

Condenser Temperature Set Point

Key process metrics for the spray drying process, which need to be monitored and recorded over the entirety of the run time, are:

Solution Feed Rate

Outlet Temperature

Cyclone Pressure Differential and Drying Gas Flow Rate

Table 35 defines spray drying process parameters/metrics, settings/ranges, and target guidelines.

TABLE 35
Spray drying variables, settings, and targets

| Variable | Setting/Range |
|---|---|
| Atomizer Installed | Spray Systems SK-MFP |
| Solution Feedrate | 130-180 kg/hr |
| Feed Pressure | 40-65 bar |
| Outlet Temperature | 22-29° C. |
| Cyclone Pressure | 10.0-12.5 cm H₂0 |
| Differential | |

### Materials

All excipients and process solvents used comply with the current monographs of the European Pharmacopoeia, the Japanese Pharmacopoeia or the USP/NF. All excipients and process solvents are purchased from approved suppliers. Manufacturer certificate of analysis are accepted and all materials received undergo testing.

TABLE 36
Materials
Material

VX-950
Hypromellose Acetate
Succinate, NF/JPE
(HPMCAS) (Aqoat AS-HG)
Sodium Lauryl Sulfate (SLS),
NF
Methylene Chloride, NF
Acetone, NF
DI Water

### Variations in Manufacture

The manufactures utilize a 10% or 30 wt % solution. Also, the solution manufacture can be varied. In some batches, the SLS/DI Water mixture is added last to the main solution reactor. Inlet temperature of the spray dryer is monitored but in some manufactures a range or a target is not defined. Reduced in-process sampling is instructed. KF testing on the polymer prior to charging can be performed.

### Other Embodiments

While a number of embodiments and examples of this invention are described herein, it is apparent that these embodiments and examples may be altered to provide additional embodiments and examples which utilize the pharmaceutical formulations and drug regimens of this invention. Therefore, it will be appreciated that the scope of this invention is to be defined by the appended claims rather than by the specific embodiments that have been represented by way of example above.

What is claimed is:

1. A therapeutic regimen comprising administering to a patient pegylated interferon alfa-2a, ribavirin and VX-950, wherein VX-950 is administered in an amount of 750 mg every eight hours, pegylated interferon alfa-2a is administered in an amount of 180 μg per week and ribavirin is administered in an amount of 1000 to 1200 mg per day.

2. The method of claim 1, wherein pegylated interferon alfa-2a, ribavirin and VX-950 are administered in an initial phase and pegylated interferon alfa-2a and ribavirin are administered over a secondary phase, wherein the secondary phase occurs after the initial phase.

3. The method of claim 1, wherein the patient is a treatment naïve patient.

4. The method of claim 1, wherein the patient is a Latino patient.

5. The method of claim 1, wherein the patient is an African American patient.

6. The therapeutic regimen of claim 2, wherein the secondary phase extends for a period of less than or about 36 weeks.

7. The therapeutic regimen of claim 6, wherein the initial phase extends for a period of less than 24 weeks.

8. The therapeutic regimen of claim 7, wherein the initial phase extends for a period of about 12 weeks.

9. The therapeutic regimen of claim 6, wherein the secondary phase extends for a period of less than 24 weeks.

10. The therapeutic regimen of claim 9, wherein the secondary phase extends for a period of about 12 weeks.

11. A therapeutic regimen comprising administering to a Latino patient pegylated interferon alfa-2a, ribavirin and VX-950, wherein VX-950 is administered in an amount of 750 mg every eight hours, pegylated interferon alfa-2a is administered in an amount of 180 μg per week and ribavirin is administered in an amount of 1000 to 1200 mg per day.

12. A therapeutic regimen comprising administering to an African American patient pegylated interferon alfa-2a, ribavirin and VX-950, wherein VX-950 is administered in an amount of 750 mg every eight hours, pegylated interferon alfa-2a is administered in an amount of 180 μg per week and ribavirin is administered in an amount of 1000 to 1200 mg per day.

13. A therapeutic regimen comprising administering to a patient pegylated interferon alfa-2a, ribavirin and VX-950 in an initial phase and administering pegylated interferon alfa-2a and ribavirin over a secondary phase, wherein the secondary phase occurs after the initial phase and VX-950 is administered in an amount of 750 mg every eight hours, pegylated interferon alfa-2a is administered in an amount of 180 μg per week and ribavirin is administered in an amount of 1000 to 1200 mg per day.

14. The therapeutic regimen of claim 13, wherein the secondary phase extends for a period of less than or about 36 weeks.

15. The therapeutic regimen of claim 14, wherein the initial phase extends for a period of less than 24 weeks.

16. The therapeutic regimen of claim 15, wherein the initial phase extends for a period of about 12 weeks.

17. The therapeutic regimen of claim 14, wherein the secondary phase extends for a period of less than 24 weeks.

18. The therapeutic regimen of claim 17, wherein the secondary phase extends for a period of about 12 weeks.

19. A therapeutic regimen comprising administering to a Latino patient pegylated interferon alfa-2a, ribavirin and VX-950 in an initial phase and administering pegylated interferon alfa-2a and ribavirin over a secondary phase, wherein the secondary phase occurs after the initial phase.

20. The method of claim 19, wherein VX-950 is administered in an amount of 750 mg every eight hours, pegylated interferon alfa-2a is administered in an amount of 180 μg per week and ribavirin is administered in an amount of 1000 to 1200 mg per day.

21. The therapeutic regimen of claim 19, wherein the secondary phase extends for a period of less than or about 36 weeks.

22. The therapeutic regimen of claim 21, wherein the initial phase extends for a period of less than 24 weeks.

23. The therapeutic regimen of claim 22, wherein the initial phase extends for a period of about 12 weeks.

24. The therapeutic regimen of claim 21, wherein the secondary phase extends for a period of less than 24 weeks.

25. The therapeutic regimen of claim 24, wherein the secondary phase extends for a period of about 12 weeks.

26. A therapeutic regimen comprising administering to an African American patient pegylated interferon alfa-2a, ribavirin and VX-950 in an initial phase and administering pegylated interferon alfa-2a and ribavirin over a secondary phase, wherein the secondary phase occurs after the initial phase.

27. The method of claim 26, wherein VX-950 is administered in an amount of 750 mg every eight hours, pegylated interferon alfa-2a is administered in an amount of 180 μg per week and ribavirin is administered in an amount of 1000 to 1200 mg per day.

28. The therapeutic regimen of claim 26, wherein the secondary phase extends for a period of less than or about 36 weeks.

29. The therapeutic regimen of claim 28, wherein the initial phase extends for a period of less than 24 weeks.

30. The therapeutic regimen of claim 29, wherein the initial phase extends for a period of about 12 weeks.

31. The therapeutic regimen of claim 28, wherein the secondary phase extends for a period of less than 24 weeks.

32. The therapeutic regimen of claim 31, wherein the secondary phase extends for a period of about 12 weeks.

33. A therapeutic regimen comprising administering to a Latino patient pegylated interferon alfa-2a, ribavirin and VX-950 in an initial phase and administering pegylated interferon alfa-2a and ribavirin over a secondary phase, wherein the secondary phase occurs after the initial phase and extends for a period of less than or about 36 weeks.

34. The therapeutic regimen of claim 33, wherein the initial phase extends for a period of less than 24 weeks.

35. The therapeutic regimen of claim 34, wherein the initial phase extends for a period of about 12 weeks.

36. The therapeutic regimen of claim 33, wherein the secondary phase extends for a period of less than 24 weeks.

37. The therapeutic regimen of claim 36, wherein the secondary phase extends for a period of about 12 weeks.

38. A therapeutic regimen comprising administering to an African American patient pegylated interferon alfa-2a, ribavirin and VX-950 in an initial phase and administering pegylated interferon alfa-2a and ribavirin over a secondary phase, wherein the secondary phase occurs after the initial phase and extends for a period of less than or about 36 weeks.

39. The therapeutic regimen of claim 38, wherein the initial phase extends for a period of less than 24 weeks.

40. The therapeutic regimen of claim 39, wherein the initial phase extends for a period of about 12 weeks.

41. The therapeutic regimen of claim 38, wherein the secondary phase extends for a period of less than 24 weeks.

42. The therapeutic regimen of claim 41, wherein the secondary phase extends for a period of about 12 weeks.

43. The method of claim 11, wherein a sustained viral response is achieved.

44. The method of claim 12, wherein a sustained viral response is achieved.
